(19) United States
(12) Patent Application Publication
(10) Pub. No.: US 2020/0215208 A1
(43) Pub. Date: Jul. 9, 2020

(54) COMPOSITIONS COMPRISING MELANIN, AND METHODS OF PREPARING AND USES THEREOF

(71) Applicants: Melanis Co., Ltd., Seoul (KR); Seoul National University R&DB Foundation, Seoul (KR); Research & Business Foundation, Sungkyunkwan University, Suwon-si (KR)

(72) Inventors: Kuk-Youn JU, Seoul (KR); Jin-Kyu LEE, Hanam-si (KR); Jung Hee LEE, Seoul (KR); Won Jae LEE, Seoul (KR)

(21) Appl. No.: 16/633,444

(22) PCT Filed: Jul. 25, 2017
(86) PCT No.: PCT/IB2017/054518

§ 371 Date: Jan. 23, 2020

(51) Int. Cl. A61K 49/18; A61K 49/12

(52) U.S. Cl. CPC A61K 49/1878; A61K 49/12

(57) ABSTRACT

The invention is directed to compositions comprising disassembled, stacked melanin oligomers, and methods of preparing and using such compositions.

### BACKGROUND OF THE INVENTION

### Field of the Invention

The invention is directed to compositions comprising disassembled, stacked melanin oligomers, and methods of preparing and using the compositions.

### Background Art

Melanins are biomacromolecules that are widely distributed in many parts of living organisms such as plants, animals, and protista, and are usually categorized into black-brown eumelanins and yellow-reddish pheomelanins. Eumelanins are derived from 3,4-dihydroxy-L-phenyl alanine (L-DOPA) or 2-(3,4-dihydroxyphenyl)ethylamine (dopamine), and pheomelanins are derived from L-DOPA or dopamine in the presence of thiol group (—SH)-containing compounds such as cysteine and glutathione.

Melanins can be obtained from natural sources or by artificial synthetic methods using enzymes or oxidants.

Many studies have been actively conducted on melanins and their methods of synthesis and applications because of their various biological functions as well as the function of blocking UV radiation as a pigment.

Melanins have been reported to have a diverse number of biological functions, including photoprotection by absorbing a broad range of electromagnetic radiation, photosensitization, metal ion chelation, antibiotic, thermoregulation, and free radical quenching. Melanins are widely used in various fields such as photovoltaic cells, sensors, optoelectric and energy storage, photoactive and photoprotective materials, antioxidant materials, biomedical applications, and cosmetics.

There is a continued need for improved melanins having improved properties.

### BRIEF SUMMARY OF THE INVENTION

Disclosed herein is a composition comprising disassembled, stacked melanin oligomers comprising 5,6-dihydroxylindole (DHI). In some embodiments, the disassembled, stacked oligomers further comprise pyrrole-2,3-dicarboxylic acid (PDCA). In some embodiments, the disassembled, stacked melanin oligomers are generated by disassembly of MelNPs.

In some embodiments, the disassembled, stacked melanin oligomers comprise 2 to about 30 layers. In some embodiments, the disassembled, stacked melanin oligomers have a thickness of about 0.3 nm to about 16 nm.

In some embodiments, the disassembled, stacked melanin oligomers are covalently bonded to poly(ethylene glycol) (PEG). The PEG can have, for example, a weight average molecular weight of about 0.3 KDa to about 40 KDa.

In some embodiments, the disassembled, stacked melanin oligomers are complexed with a paramagnetic metal ion. The paramagnetic metal ion can be, for example, gadolinium (Gd), iron (Fe), manganese (Mn), nickel (Ni), copper (Cu), erbium (Er), europium (Eu), holmium (Ho), and/or chromium (Cr).

In some embodiments, the composition can further comprise a magnetic resonance imaging (MRI) contrast agent.

In some embodiments, the composition is a pharmaceutical composition.

Disclosed herein is a method of preparing a composition comprising disassembled, stacked melanin oligomers, comprising: adding a base to melanin-like nanoparticles (MelNPs) comprising 5,6-dihydroxylindole (DHI) and disassembling the MelNPs into disassembled, stacked melanin oligomers; and adding an acid to neutralize the disassembled, stacked melanin oligomers. In some embodiments, the disassembling occurs at pH 9 or greater.

Also disclosed herein is a method of preparing a composition comprising disassembled, stacked melanin oligomers, comprising: adding a base to melanin-like nanoparticles (MelNPs) comprising 5,6-dihydroxylindole (DHI) to obtain a pH of greater than 10.5 and disassembling the MelNPs into disassembled, stacked melanin oligomers.

In some embodiments, the MelNPs further comprise pyrrole-2,3-dicarboxylic acid (PDCA). In some embodiments, the MelNPs are synthesized from a melanin precursor of dopamine. In some embodiments, the method is performed under a deoxygenated and/or nitrogen purged condition. In other embodiments, the method is performed under an oxygenated condition.

In some embodiments, the composition is prepared in the presence of poly(ethylene glycol) (PEG). The PEG can have, for example, a weight average molecular weight of about 0.3 KDa to about 40 KDa.

In some embodiments, the disassembled, stacked melanin oligomers comprise 2 to about 30 layers. In some embodiments, the disassembled, stacked melanin oligomers have a thickness of about 0.3 nm to about 16 nm.

In some embodiments, the method can further comprise adding a paramagnetic metal ion to the disassembled, stacked melanin oligomers. The paramagnetic metal ion can be, for example, gadolinium (Gd), iron (Fe), manganese (Mn), nickel (Ni), copper (Cu), erbium (Er), europium (Eu), holmium (Ho), and/or chromium (Cr).

Also disclosed herein is a method of imaging a subject, comprising: exposing a subject to an imaging device, wherein the composition disclosed herein has been introduced to the subject; and detecting the disassembled, stacked melanin oligomers that are selectively accumulated in an area. Also disclosed herein is a method of imaging a disease in a subject, comprising: exposing a subject to an imaging device, wherein the composition disclosed herein has been introduced to the subject; and detecting the disassembled, stacked melanin oligomers that are selectively accumulated in a disease area. In some embodiments, the disease is a tumor. In some embodiments, the imaging device is MRI.

### BRIEF DESCRIPTION OF THE DRAWINGS/FIGURES

FIG. 1. Experimental scheme 1. pH-controlled disassembly process for melanin-like nanoparticles (MelNPs).

FIG. 2. TEM images of (A) protomolecules resulting from disassembly of MelNPs and (B) oxidized protomolecules generated by pH-controlled disassembly process in presence of oxygen; (C) AFM images of (A) protomolecules resulting from disassembly of MelNPs and (B) oxidized protomolecules generated by disassembly process in presence of oxygen; (E) height analysis of protomolecules and (F) oxidized protomolecules generated from disassembly of MelNPs.

FIG. 3. Experimental scheme 2.

FIG. 4. (A) Dispersion stability of PEG-protomolecules-Fe³⁺ in PBS. After chelation with Fe³⁺ ions, PEG-protomolecules-Fe³⁺ and PEG-oxidized protomolecules-Fe³⁺ showed stable dispersion behavior in PBS. (B) Hydrodynamic radius of PEG-protomolecules-Fe³⁺ and PEG-oxidized protomolecules-Fe³⁺.

FIG. 5. T1 MR images of (A) Fe³⁺-MelNPs (˜100 nm radius)-PEG, (B) PEG-protomolecules-Fe³⁺, (C) PEG-oxidized protomolecules-Fe³⁺ with variable concentration.

FIG. 6. T1 weighted MRI of mouse bearing liver tumor after injection of PEG-MelNPs-Fe³⁺, PEG-protomolecules-Fe³⁺ and PEG-oxidized protomolecules-Fe³⁺.

FIG. 7. T1 weighted MRI of mouse bearing liver tumor after injection of PEG-oxidized protomolecules-Fe³⁺.

FIG. 8. Schematic illustration of pH-controlled disassembly and simultaneous disassembly/oxidation of hierarchically assembled Sepia eumelanin.

FIG. 9. (a) Experimental scheme of pH-controlled disassembly (Black pathway) and simultaneous disassembly/oxidation process (Blue pathway) for Sepia eumelanin, (b) TEM images of Sepia eumelanin, (c) partially disassembled Sepia particles, (d) subunits disassembled from Sepia particles and (e) oxidized subunits. (f) Tapping-mode AFM height image and (g) height histogram of non-oxidized subunits of Sepia eumelanin after the removal of dissolved salts and oligomer species of MW<2000 by dialysis. (h) CP-MAS ¹³C solid-state NMR spectrum of oxidized subunits with comparison to parental Sepia and its non-oxidized subunits. The bottom column shows magnified spectra ranging from 150 to 220 ppm. The arrow indicates characteristic peak around ranging 180-185 ppm corresponding to carbonyl resonances of pyrrole carboxylic acid resulting from oxidative partial degradation of Sepia subunits. (i) Tapping-mode AFM height image and (j) height histogram of oxidized subunits after the removal of dissolved salts and oxidized oligomers MW<2000.

FIG. 10. (a) TEM images of synthetic melanin-like nanoparticles (MelNPs), (b) partially disassembled MelNPs, and (c) subunits disassembled from MelNPs.

FIG. 11. UV-vis absorption properties of Sepia eumelanin as a function of structural alteration. (a) UV-vis absorption spectra of Sepia subunits and oxidized subunits with respect to their parental particles; the weight concentration of each solution is equivalent. (b) UV-vis absorption spectra of subunits resulting from pH-controlled disassembly of Sepia before and after removing the oligomeric unit fraction (MW<2000) through dialysis. (c) UV-vis absorption spectra of oxidized subunits resulting from simultaneous disassembly/oxidation of Sepia before and after removing oxidized oligomeric unit fraction (MW<2000) through dialysis.

FIG. 12. Emission spectra of Sepia eumelanin as a function of structural alteration. (a) UV-vis absorption and (b) emission spectra of Sepia subunits (Subunits), oxidized subunits (Ox-subunits) and parental particles (Sepia). Subunits were obtained by disassembly of Sepia and Ox-subunits were collected by simultaneous disassembly/oxidation of Sepia. For emission spectra, the absorbance of all samples at 314 nm was tuned to be equivalent by adjusting the concentration as shown in (a). Emission spectra were corrected with excitation at 314 nm. The concentration of each sample was highly diluted until the absorbance at the wavelength matching a sample's emission peak was below 0.1 to minimize fluorescence reabsorption. The inset shows emission spectra of Sepia and its subunits and oxidized subunits at the equivalent weight concentration. It also shows the emission intensity as a function of structural alteration. (c) UV-vis absorption and (d) emission spectra of size-selected subunits and oxidized subunits. In a similar manner, the absorbance of all samples at 314 nm was tuned to be equivalent by adjusting the concentration for collecting emission spectra at an excitation of 314 nm. The concentration of each sample was highly diluted until the absorbance at the wavelength matching a sample's emission peak was below 0.1 to minimize fluorescence reabsorption. (e) Normalized excitation spectra corresponding to emission spectra shown in (c). Excitation spectra of size-selected subunits and oxidized subunits were taken at the maximum wavelength of each emission peak. The excitation spectra show a characteristic peak ranging from 314 to 355 nm. The spectra are normalized at the peaks around 314-355 nm. f) Normalized emission spectra of oligomeric Sepia subunit fraction before and after oxidation. Note that the isolated oligomeric unit fraction was selectively oxidized by increasing pH in oxygen-dissolved water. The absorbance at 314 nm for the oligomeric fraction and the oxidized product was tuned to be equivalent by adjusting the concentration. The spectra were corrected with excitation at 314 nm. The inset shows unnormalized emission spectra, which indicates that the emission intensity of oligomeric subunit fraction is slightly decreased with oxidation. (g) Normalized excitation spectra of the oligomeric subunit fraction and the oxidized product. The spectra were taken at the maximum wavelength of each emission peak. The spectra are normalized at the peak near 314 nm.

FIG. 13. (a) Time profile of the relative amount of photo-generated superoxide radical by oxidized Sepia subunits compared with their parental particles, non-oxidized subunits and oxidized subunit fraction (MW>2000) composed of stacked oligomers and (b) their comparison results under non-irradiation conditions. (c) Time profile of the relative amount of photo-generated hydroxyl radical by oxidized Sepia subunits compared with their parental particle, non-oxidized subunits and oxidized subunit fraction (MW>2000) composed of stacked oligomers and (d) their control experiment results under non-irradiation conditions.

FIG. 14. Proposed mechanism of the Janus behavior of eumelanin.

FIG. 15. TEM images of partially disassembled sepia eumelanin during pH-controlled disassembly with (a) pH 9.5 and (b) pH 12.5.

### DETAILED DESCRIPTION OF THE INVENTION

### Compositions, and Methods of Preparing and Uses Thereof

As used herein, the term “melanin” means biomacromolecules that are distributed in many parts of living organisms such as plants, animals, and protista, and is usually categorized into black-brown eumelanins and yellow-reddish pheomelanins. Eumelanins are derived from 3,4-dihydroxy-L-phenyl alanine (L-DOPA) or 2-(3,4-dihydroxyphenyl)ethylamine (dopamine), and pheomelanins are derived from L-DOPA or dopamine in the presence of thiol group (—SH)-containing compounds such as cysteine and glutathione. Eumelanins are black pigments that are predominantly found in mammals.

Melanin nanoparticles can be obtained from natural sources or by chemical synthetic methods (melanin-like nanoparticles or MelNPs). When obtained from natural sources, they can be recovered, e.g., from the ink of cuttlefish by centrifugation. When synthesized by chemical methods, they can be synthesized from a melanin precursor of, e.g., dopamine, DOPA, cysteine, or tyrosine.

As used herein, the term “melanin nanoparticles” or “melanin-like nanoparticles” refers to very small melanin particles having a nanoscale diameter. For example, the size can be a mean or median diameter in a range of about 30 nm to about 600 nm, about 30 nm to about 400 nm, about 30 nm to about 200 nm, or about 50 nm to about 100 nm. Methods for preparing melanin or melanin-like nanoparticles are known in the art and results in aggregated or polymers of melanin nanoparticles. See, e.g., U.S. Pat. No. 8,937,149, US2015/0139914, and US2014/0356284, each of which is herein incorporated by reference in its entirety.

The present disclosure provides compositions comprising disassembled, stacked melanin oligomers comprising 5,6-dihydroxylindole (DHI). In some embodiments, the oligomers further comprise pyrrole-2,3-dicarboxylic acid (PDCA). In some embodiments, the compositions comprising disassembled, stacked melanin oligomers comprising DHI are generated by disassembly of MelNPs. The MelNPs can comprise DHI or a combination of DHI and PDCA.

Also provided herein is a method of preparing a composition comprising disassembled, stacked melanin oligomers that are produced by disassembly of MelNPs comprising 5,6-dihydroxylindole (DHI). Also provided is a method of preparing a composition comprising disassembled, stacked melanin oligomers, the method comprising: adding a base to melanin-like nanoparticles (MelNPs) comprising 5,6-dihydroxylindole (DHI) and disassembling the MelNPs into stacked oligomers; and adding an acid to neutralize the disassembled, stacked melanin oligomers. Also provided is a method of preparing a composition comprising disassembled, stacked melanin oligomers, the method comprising: adding a base to melanin-like nanoparticles (MelNPs) comprising 5,6-dihydroxylindole (DHI) to obtain a pH of greater than 10.5 and disassembling the MelNPs into stacked melanin oligomers. In some embodiments, the MelNPs further comprise pyrrole-2,3-dicarboxylic acid (PDCA).

As used herein, the term “disassembled” or “disassembly” means that the assembled or assembly structure of melanin nanoparticles or MelNPs are disintegrated into or reduced in size to “stacked melanin oligomers.” In some embodiments, the compositions disclosed herein are nonpolymeric or deaggregated. It has been discovered that the melanin product generated by oxidation of 5,6-dihydroxyindole (DHI) is composed of planar oligomers and they are prone to stacking because of their planar structure; the stacked oligomers are aggregated to form the particle character of the melanin disclosed herein. In contrast, melanin products produced by oxidation of 5,6-dihydroxyindole-2-carboxylic acid (DHICA) are composed of non-planar oligomers and they are prone to aggregate without stacking to form the particle character of melanin. Because MelNPs disclosed herein is synthesized by spontaneous oxidation of dopamine, fundamental oligomer units of MelNPs are DHI and optionally the oxidized form of DHI, such as pyrrole-2,3-dicarboxylic acid (PDCA). Therefore, MelNPs generated by oxidation of dopamine is composed of highly stacked oligomers. See Panzella et al., Angew. Chem. Int. Ed. 52:12684-12687 (2013), and Yu et al., Langmuir 30:5497-5505 (2014), each of which is herein incorporated by reference. In some embodiments, the stacked melanin oligomers are predominantly DHI, or a combination of DHI and PDCA, e.g., 70% or greater, 80% or greater, 90% or greater, 95% or greater, 98% or greater, or 99% or greater.

As used herein, a “polymer” is a molecule of higher molecular weight composed of many repetition of monomer units. As used herein, an “oligomer” is a molecule composed of about 3 to about 13 monomer units. In some embodiments, the compositions disclosed herein are substantially nonpolymeric or deaggregated.

In some embodiments, the disassembled, stacked melanin oligomers comprise 2 to about 30 oligomer layers, 2 to about 20 oligomer layers, or 2 to about 15 oligomer layers.

The thickness of one oligomer is about 0.15 to about 0.2 nm. The inter-stacking distance between 2 oligomers is about 0.34 nm. In some embodiments, the thickness of the stacked oligomers is about 0.3 to about 16 nm, about 0.3 to about 10 nm, or about 0.3 to about 8 nm, about 0.3 to about 6 nm, or about 0.3 to about 5 nm.

In some embodiments, a base can be added to achieve a basic condition, e.g., a pH of about 9 or greater, a pH of about 10 or greater, a pH of greater than 10, a pH of great than 10.5, or a pH of greater than 10.5 to about 14.

Non-limiting examples of a base are alkali metal hydroxides, alkaline earth metal hydroxides, alkali metal carbonates, alkaline earth metal carbonates, alkali metal bicarbonates, alkaline earth metal bicarbonates, alkali metal acetates, alkali metal phosphates, alkali metal alkoxides (1-20 carbon atoms), ammonia (NH₃), ammonium hydroxide (NH₄OH), amine or the like. In some embodiments, the base can be NaOH, NH₄OH, KOH, Ca(OH)₂, LiOH, K₂CO₃, methylamine, ethylamine, and/or diethylamine.

In some embodiments, an acid can be added to neutralize the basic condition, e.g., to stop the disassembling reaction.

Non-limiting examples of an acid are inorganic acids, such as hydrogen halides and their solutions: hydrofluoric acid (HF), hydrochloric acid (HCl), hydrobromic acid (HBr), hydroiodic acid (HI); sulfuric acid (H₂SO₄); nitric acid (HNO₃); phosphoric acid (H₃PO₄); and carboxylic acids, such as acetic acid (CH₃COOH), citric acid (C₆H₈O₇), formic acid (HCOOH), gluconic acid (HOCH₂—(CHOH)₄—COOH), lactic acid (CH₃—CHOH—COOH), oxalic acid (HOOC—COOH), and tartaric acid (HOOC—CHOH—CHOH—COOH). In some embodiments, the acid is KH₂PO₄.

In some embodiments, the composition comprising disassembled, stacked melanin oligomers is prepared under oxygenated conditions. Alternatively, the composition is prepared under deoxygenated and/or nitrogen purged conditions.

In some embodiments, the disassembled, stacked melanin oligomers can be coordinated with metals at their functional groups such as carbonyl, amine, imine, phenol, and O-diphenol.

In some embodiments, the disassembled, stacked melanin oligomers can be surface modified by adding, e.g., thiol (—SH)-terminated alkoxy polyethylene glycol. Specifically, when the disassembled, stacked melanin oligomers are surface-modified with thiol-terminated alkoxy polyethylene glycol, alkoxy polyethylene glycol binds to the disassembled, stacked melanin oligomers by 1,4-addition reaction of the nucleophilic thiol group with quinone of oligomers, resulting in surface modification of the disassembled, stacked melanin oligomers.

In addition, thiol-terminated alkoxy polyethylene glycol can have a weight average molecular weight of approximately 300-40000 Da.

The surface-modified disassembled, stacked melanin oligomers can be readily dispersed in biological media, thereby being applied to various fields, in particular, biological fields. Non-limiting examples of the biological media are phosphate buffer solution (PBS), fetal bovine serum (FBS) or the like.

Provided herein is a method of imaging a subject, comprising: exposing a subject to an imaging device, wherein the composition comprising disassembled, stacked melanin oligomers comprising DHI has been introduced to the subject; and detecting the disassembled, stacked melanin oligomers that are selectively accumulated in an area. Also provided is a method of imaging a disease in a subject, comprising: exposing a subject to an imaging device, wherein the composition comprising disassembled, stacked melanin oligomers comprising DHI has been introduced to the subject; and detecting the disassembled, stacked melanin oligomer that is selectively accumulated in a disease area. In some embodiments, the disassembled, stacked melanin oligomers further comprise PDCA. In some embodiments, the disease is a tumor. In some embodiments, the imaging device is MRI.

In some embodiments, the disclosure provides a contrast agent for magnetic resonance imaging (MRI or MR), including a composition comprising disassembled, stacked melanin oligomers, wherein the composition has stable dispersibility in water. In some embodiments, paramagnetic metal ions are coordinated to the disassembled, stacked melanin oligomers. In some embodiments, PEGS are attached to the disassembled, stacked melanin oligomers.

As used herein, the term “magnetic resonance image” or “nuclear magnetic resonance image” means imaging based on the nuclear magnetic resonance phenomenon which occurs due to absorption of the energy during the transition to another energy level by action of a particular external energy on a magnetic moment of atomic nucleus in a magnetic field.

The disclosure herein provides a contrast agent for magnetic resonance imaging, which is characterized in that the disassembled, stacked melanin oligomers described herein are used as target specific contrast agent when they are complexed with paramagnetic metal ions without any surface modification capable of target specific imaging.

Further, the contrast agent for magnetic resonance imaging of the present invention is characterized in that the paramagnetic metal ions form coordinate bonds with the disassembled, stacked melanin oligomers.

As used herein, the term “paramagnetic metal ion” means a material showing magnetic resonance image, in which internal unpaired spins are randomly oriented due to thermal motion, but in a magnetic field, the spins can align to a predetermined direction. That is, it means a material that retains no magnetism as usual, but it is magnetized toward the magnetic field when an external magnetic field is applied. Examples thereof can include ions of one or more metals selected from the group consisting of gadolinium (Gd), iron (Fe), manganese (Mn), nickel (Ni), copper (Cu), erbium (Er), europium (Eu), holmium (Ho) and chromium (Cr).

The paramagnetic metal ion can form a coordinate bond with the disassembled, stacked melanin oligomers. When the paramagnetic metal ion is coordinated to the disassembled, stacked melanin oligomers, it shows a stronger T1 shortening effect than MelNPs that are complexed with paramagnetic metal ion, thereby exhibiting an excellent contrast effect of nuclear magnetic resonance imaging in T1-weighted images.

Further, the contrast agent for magnetic resonance imaging of the present invention is characterized in that disassembled, stacked melanin oligomers generated by disassembly of MelNPs in the presence of oxygen show T1 contrast enhanced capability when they are complexed with paramagnetic metal ion such as ferric ion. In the case of disassembled, stacked melanin oligomers generated by disassembly of MelNPs in the presence of dissolved oxygen, they showed higher T1 contrast enhanced capability than MelNPs (100 nm). When they are injected into mouse bearing liver tumor via tail vain, they showed selective contrast enhancement in the tumor after 24 h injection. Through selective T1 contrast enhancement in liver tumor, presence of tumor in liver can be determined.

Further, the present invention provides a method for preparing the contrast agent for magnetic resonance imaging, comprising: adding paramagnetic metal ions to a composition comprising disassembled, stacked melanin oligomers to form coordinate bonds between the paramagnetic metal ions and the disassembled, stacked melanin oligomers; adding PEGS to the mixture of paramagnetic metal ions and disassembled, stacked melanin oligomers; and recovering the prepared contrast agent. The disassembled, stacked melanin oligomers, paramagnetic metal ions, and PEGS are the same as described above.

For example, a solution containing the paramagnetic metal ions can be added to a composition comprising disassembled, stacked melanin oligomers, and then stirred for approximately 3 to 10 min to form coordinate bonds. The disassembled, stacked melanin oligomers prepared with paramagnetic metal ions can be recovered by centrifugation and then dispersed in water.

The contrast agent for magnetic resonance imaging according to the present invention has no cytotoxicity, and a long retention time in vivo, compared to the conventional contrast agent for nuclear magnetic resonance imaging, thereby being usefully applied as an MRI contrast agent. In some embodiments, the contrast agent for nuclear magnetic resonance imaging according to the present invention showed excellent r₂/r₁, compared to Fe₂O₃, MnO, Hollow Mn₃O₄, and showed r₂/r₁ similar to that of Gd-DTPA. Further, Gd-BTPA shows a snort contrast effect whereas the contrast agent for nuclear magnetic resonance imaging according to the present invention has a long retention time in vivo and therefore, it is more effective to secure the time taken for attachment to particular tissues or cells.

Contrast agents can be injected intravenously to enhance the appearance of blood vessels, tumors or inflammation. MRI is used to image every part of the body, but is particularly useful in neurological conditions, disorders of the muscles and joints, for evaluating tumors and showing abnormalities in the heart and blood vessels.

The term “sample” can refer to a tissue sample, cell sample, a fluid sample, and the like. The sample can be taken from a subject. The tissue sample can include brain, hair (including roots), buccal swabs, blood, saliva, semen, muscle, or from any internal organs, or cancer, precancerous, or tumor cells associated with any one of these. The fluid can be, but is not limited to, urine, blood, ascites, pleural fluid, spinal fluid, and the like. The body tissue can include, but is not limited to, brain, skin, muscle, endometrial, uterine, and cervical tissue or cancer, precancerous, or tumor cells associated with any one of these. In some embodiments, the body tissue is brain tissue or a brain tumor or cancer.

The term “administration” refers to introducing a composition of the present disclosure into a subject. In some embodiments, the route of administration of the composition is oral administration. In some embodiments, the route of administration is intravenous administration. However, any route of administration, such as topical, subcutaneous, peritoneal, intraarterial, inhalation, vaginal, rectal, nasal, introduction into the cerebrospinal fluid, or instillation into body compartments can be used.

As used herein, the term “host,” “subject,” or “patient,” includes humans, mammals (e.g., mice, rats, pigs, cats, dogs, and horses), and poultry. Typical hosts to which compositions of the present disclosure can be administered are mammals, particularly primates, non-humans or humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted above or another organism that is alive. The term “living subject” refers to the entire subject or organism and not just a part excised (e.g., a liver or other organ) from the living subject.

A polyethylene glycol (PEG)-disassembled, stacked melanin oligomers can include a PEG attached to each melanin oligomer.

In some embodiments, the PEG can be bonded (e.g., directly or indirectly) to the melanin oligomer. For example, the PEG can be bonded to the melanin oligomer via thiol or amine groups on the PEG. In some embodiments, the PEG-melanin nanoparticle can include 5 to 50 PEGS. In an embodiment, the PEG can be a linear PEG, a multi-arm PEG, a branched PEG, and combinations thereof. The weight average molecular weight of the PEG can be about 0.3 kDa to about 40 kDa, about 1 kDa to 40 kDa, about 1 kDa to 30 kDa, about 1 kDa to 20 kDa, about 1 kDa to 12 kDa, about 1 kDa to 10 kDa, or about 1 kDa to 8 kDa. When used in reference to PEG moieties, the word “about” indicates an approximate average molecular weight and reflects the fact that there will normally be a certain molecular weight distribution in a given polymer preparation.

Alternatively, one or more PEGS can be replaced with n-MEG, poly(vinyl alcohol) (PVA), poly(acrylic acid) (PAA), poly(propylene furmarate-co-ethylenee glycol) (P(PF-co-EG)), polyacrylamide, polypeptides, poly-N-substituted glycine oligomers (polypeptoids), and the like, as well as with naturally derived polymers normally include hyaluronic acid (HA), alginate, chitosan, agarose, collagen, fibrin, gelatin, dextran, and any combination thereof, as well as derivatives of each of these.

In some embodiments, the PEG-disassembled, stacked melanin oligomers can include a MRI agent that has a detectable MRI signal. In some embodiments, the amount or number of MRI agents disposed (e.g., directly or indirectly) on the PEG-disassembled, stacked melanin oligomers can be about 1 to 50 MRI agents. In some embodiments, all or a portion of the MRI agents can be directly disposed on the PEG or the PEG-disassembled, stacked melanin oligomers. In other words, where the MRI agent is Gd, Gd can directly attached to the PEG-disassembled, stacked melanin oligomers and/or attached to the PEG via a linker compound (e.g., a chelator) such as DOTA (e.g., via a maleimide linkage (see below)). In some embodiments, all of the MRI agents are indirectly attached to the PEG-disassembled, stacked melanin oligomer surface via one or more linkers, such as DOTA.

The MRI agent can be Gd, iron oxide, paramagnetic chemical exchange saturation transfer (CEST) agents, ¹⁹F active materials, manganese, or a substance that shortens or elongates T1 or T2, and a combination thereof. The Gd MRI agent can be a compound such as DOTA-Gd, DTPA-Gd, Gd within a polymeric chelator. The iron oxide MRI agent can be a compound such as a small paramagnetic iron oxide (SPIO) or an ultrasmall SPIO with or without a dextran or other stabilizing layer. The paramagnetic CEST MRI agent can be a compound such as lanthamide complexes.

The MRI agent can be linked to the PEG surface via a linkage such as a maleimide linkage, NHS ester, click chemistry, or another covalent or non-covalent approach, or a combination thereof.

In some embodiments, the PEG-melanin oligomers do not require a raiolabel for imaging. In alternative embodiments, the PEG-melanin nanoparticle can include a radiolabel for imaging. In an exemplary embodiment, the radiolabel can include one or more of the following: ⁶⁴Cu, ¹²⁴I, ^{76/77}Br, ⁸⁶Y, ⁸⁹Zr, ⁶⁸Ga, ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, or ¹⁵³Sm. Furthermore, the PEG-disassembled, stacked melanin oligomers can include another agent (e.g., a chemical or biological agent), where the agent can be disposed indirectly or directly on the PEG-melanin oligomers. In particular, the probe can include, but is not limited to, a drug, a therapeutic agent, a radiological agent, a chemological agent, a small molecule drug, a biological agent (e.g., peptides, proteins, antibodies, antigens, and the like) and combinations thereof, that can be used to image, detect, study, monitor, evaluate, treat, and/or screen a disease, condition, or related biological event corresponding to the target. In some embodiments, the agent is included in an effective amount to accomplish its purpose (e.g., therapeutically effective amount).

In some embodiments, the steps of this method can be repeated at determined intervals so that the location and/or size of the disease can be monitored as a function of time and/or treatment. In particular, the PEG-disassembled, stacked melanin oligomer can find use in a host undergoing chemotherapy or other treatment (e.g., using a drug), to aid in visualizing the response of a disease or tumor to the treatment. The PEG-disassembled, stacked melanin oligomer is typically visualized and sized prior to treatment, and periodically (e.g., daily, weekly, monthly, intervals in between these, and the like) during chemotherapy, radiotherapy, and the like, to monitor the tumor size. Other labeled probes can be used in a similar manner.

It should be noted that the amount effective to result in uptake of the composition comprising disassembled, stacked melanin oligomer or PEG-modified composition thereof into the cells or tissue of interest can depend upon a variety of factors, including for example, the age, body weight, general health, sex, and diet of the host; the time of administration; the route of administration; the rate of excretion of the specific probe employed; the duration of the treatment; the existence of other drugs used in combination or coincidental with the specific composition employed; and like factors well known in the medical arts.

### Dosage Forms

Embodiments of the present disclosure can be included in one or more of the dosage forms mentioned herein. Unit dosage forms of the pharmaceutical compositions (the “composition” includes at least the composition comprising disassembled, stacked melanin oligomers that are labeled, e.g., with PEG, can be suitable for oral, mucosal (e.g., nasal, sublingual, vaginal, buccal, or rectal), parenteral (e.g., intramuscular, subcutaneous, intravenous, intra-arterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the present disclosure typically vary depending on their use. For example, a parenteral dosage form can contain smaller amounts of the active ingredient than an oral dosage form used to treat the same condition or disorder. These and other ways in which specific dosage forms encompassed by this disclosure vary from one another will be readily apparent to those skilled in the art (see, e.g., Remington's Pharmaceutical Sciences, 18th ed., Mack Publishing, Easton, Pa. (1990)).

Typical compositions and dosage forms of the compositions of the disclosure can include one or more excipients. Suitable excipients are well known to those skilled in the art of pharmacy or pharmaceutics, and non-limiting examples of suitable excipients are provided herein. Whether a particular excipient is suitable for incorporation into a composition or dosage form depends on a variety of factors well known in the art including, but not limited to, the way in which the dosage form will be administered to a patient. For example, oral dosage forms, such as tablets or capsules, can contain excipients not suited for use in parenteral dosage forms. The suitability of a particular excipient can also depend on the specific active ingredients in the dosage form. For example, the decomposition of some active ingredients can be accelerated by some excipients, such as lactose, or by exposure to water. Active ingredients that include primary or secondary amines are particularly susceptible to such accelerated decomposition.

The disclosure encompasses compositions and dosage forms of the disclosure herein that can include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure can contain one or more solubility modulators, such as sodium chloride, sodium sulfate, sodium or potassium phosphate, or organic acids. An exemplary solubility modulator is tartaric acid.

A “pharmaceutical composition,” as used herein, refers to a composition comprising disassembled, stacked melanin oligomers with other chemical components that are pharmaceutically acceptable, such as but not limited to carriers, stabilizers, diluents, disintegrants, suspending agents, thickening agents, binders, antimicrobial agents, antimicrobial preservatives, antioxidants, and/or buffering agents. The pharmaceutical composition facilitates administration of the calcium lactate to a subject.

“Pharmaceutically acceptable salt” refers to those salts that retain the biological effectiveness and properties of the free bases and that are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

Embodiments of the present disclosure include pharmaceutical compositions that include the labeled probe (e.g., PEG-disassembled, stacked melanin oligomers), pharmaceutically acceptable salts thereof, with other chemical components, such as physiologically acceptable carriers and excipients. One purpose of a pharmaceutical composition is to facilitate administration of labeled probe (e.g., PEG-disassembled, stacked melanin oligomer) to a subject (e.g., human).

Embodiments of the present disclosure can be salts and these salts are within the scope of the present disclosure. Reference to a compound of any of the formulas herein is understood to include reference to salts thereof, unless otherwise indicated. The term “salt(s)”, as employed herein, denotes acidic and/or basic salts formed with inorganic and/or organic acids and bases. In addition, when an embodiment of the present disclosure contains both a basic moiety and an acidic moiety, zwitterions (“inner salts”) can be formed and are included within the term “salt(s)” as used herein. Pharmaceutically acceptable (e.g., nontoxic, physiologically acceptable) salts are preferred, although other salts are also useful, e.g., in isolation or purification steps which can be employed during preparation. Salts of the compounds of an active compound can be formed, for example, by reacting an active compound with an amount of acid or base, such as an equivalent amount, in a medium such as one in which the salt precipitates or in an aqueous medium followed by lyophilization.

Embodiments of the present disclosure that contain a basic moiety can form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Embodiments of the present disclosure that contain an acidic moiety can form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like.

Basic nitrogen-containing groups can be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the compounds of the disclosure are also contemplated herein. Solvates of the compounds are preferably hydrates.

The amount of the composition comprising disassembled, stacked melanin oligomers, in a dosage form can differ depending on various factors. It will be understood, however, that the total daily usage of the compositions of the present disclosure will be decided by the attending physician or other attending professional within the scope of sound medical judgment. The specific effective dose level for any particular host will depend upon a variety of factors, including for example, the activity of the specific composition employed; the specific composition employed; the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts. For example, it is well within the skill of the art to start doses of the composition at levels lower than those required to achieve the desired effect and to gradually increase the dosage until the desired effect is achieved.

### Kits

The present disclosure also provides packaged compositions or pharmaceutical compositions comprising a pharmaceutically acceptable carrier and a composition comprising disassembled, stacked melanin oligomers of the disclosure. In certain embodiments, the packaged compositions or pharmaceutical composition includes the reaction precursors to be used to generate the labeled probe according to the present disclosure. Other packaged compositions or pharmaceutical compositions provided by the present disclosure further include indicia including at least one of: instructions for using the labeled probe to image a host, or host samples (e.g., cells or tissues), which can be used as an indicator of conditions including, but not limited to, cancers, melanin related diseases, and biological related conditions.

Embodiments of this disclosure encompass kits that include, but are not limited to, the composition comprising disassembled, stacked melanin oligomers and directions (written instructions for their use). The components listed above can be tailored to the particular biological condition to be monitored as described herein. The kit can further include appropriate buffers and reagents known in the art for administering various combinations of the components listed above to the host cell or host organism. The imaging agent and carrier can be provided in solution or in lyophilized form. When the imaging agent and carrier of the kit are in lyophilized form, the kit can optionally contain a sterile and physiologically acceptable reconstitution medium such as water, saline, buffered saline, and the like.

### EXAMPLES

### Example 1

Experimental Details

Synthesis of MelNPs.

180 mg of dopamine hydrochloride (Aldrich Chemical) was dissolved in 45 mL of deionized water. A NaOH (670 μL, 1 N) solution was added to the solution of dopamine hydrochloride under vigorous stirring at 50° C. After 5 h, MelNPs were isolated and purified by centrifugation/redispersion in deionized water (4000 rpm for 10 min) several times.

Preparation of Disassembled Stacked Melanin Oligomers (Protomolecules) and Oxidized Protomolecules from MelNPs.

3.5 mL of NaOH solution (1 M) was added into 1 mL of MelNPs suspension (1 mg/mL) under N₂ purging. MelNPs suspension and NaOH solution were pre-purged by N₂ for 20 min to eliminate dissolved O₂. After 12 h, 5 mL of deoxygenated KH₂PO₄ solution (1 M) was added into the suspension to neutralize the solution pH and prevent additional pH-induced oxidation of resulting products under ambient environment. Oxidation process of MelNPs was progressed for 5 days with same manner as described above excepting N₂ purging.

Preparation of PEG-Protomolecules-Fe³⁺.

5 mL of MelNPs (4 mg/mL) was pre-purged with nitrogen gas for 20 min for elimination of oxygen. After adding 26 mg of mPEG-SH (M.W. 2000) into the MelNP suspension, 2 mL of de-oxygenated NaOH solution (1M) was added into the suspension. After 1 h, 2.8 mL of de-oxygenated KH₂PO₄ solution (1M) was added into the suspension. Through dialysis (MWCO 2000) for 12 h, salts dissolved in the suspension was eliminated. Iron chloride solution was added into PEGylated subunits. Weight ratio between Fe³⁺ and melanin subunits was in range from 0.1:1 to 10:1. After exposure to iron chloride solution, the PEGylated proromolecule suspension was centrifuged with centrifugal tube (MWCO=3 KDa) several times.

Preparation of PEG-Oxidized Protomolecules-Fe.

After adding 26 mg of mPEG-SH (M.W. 2000) into 5 mL of MelNP suspension (4 mg/Ml), 2 mL of NaOH solution (1M) was further added. After 1 h, 2.8 mL of KH₂PO₄ solution (1M) was added into the suspension. Through dialysis (MWCO 2000) for 12 h, salts dissolved in the suspension was eliminated. Iron chloride solution was added into PEGylated oxidized subunits. Weight ratio between Fe³⁺ and melanin oxidized subunits was in range from 0.1:1 to 10:1. After exposure to iron chloride solution, the PEGylated oxidized subunit suspension was centrifuged with centrifugal tube (MWCO=3 KDa) several times.

Characterization of PEG-Protomolecules-Fe and PEG-Oxidized Protomolecules-Fe.

After dispersion in PBS for 24 h, dispersion stability of PEG-subunits-Fe and PEG-oxidized subunits-Fe was determined. They didn't show any aggregation in PBS within 7 days. Size distribution of PEG-subunits-Fe and PEG-oxidized subunits-Fe was determined by dynamic light scattering (DLS) through a particle size analyzer (Mavern, Zetasizer Nano ZS90).

Preparation of Animal Models.

Male 6-week-old BALB/C nude mice were purchases from Orient Bio (Seoul, Korea). All animal studies were approved by the institutional Animal Care and Use Committee of Samsung Biomedical Research Institute (Seoul, Korea). Orthotopic liver tumor model was created using human HCC liver tumor cell line (HepG2, ATCC). The HepG2 These cells were maintained in Minimum Essential Medium with 10% fetal bovine serum (Invitrogen) and 1% antibiotics (ThermoFisher). Cells were cultured at 37° C. and 5% CO₂, and harvested with 0.25% Trypsin/EDTA (ThermoFisher). The harvested cells (1×10⁶ HepG2 cells) suspended in 10 ul HMS with Matrigel (1:1). After sampling the cells, the mouse was fully anesthetized by breathing 2% isoflurane in mixture of O₂/Air gas (3:7 ratio) with face mask and the mouse was exposed the liver. The mixed cells with matrigel were slowly injected into the liver. After 4-6 weeks, tumor size was checked using the MR.

In Vivo MR Images.

The MR images were obtained by following method. The mice were initially anesthetized using 5% isoflurane and afterwards anesthesia was maintained with 1.5-2% isoflurane in a mixture of O²/Air gas (3:7) by using a face mask. The body temperature was maintained at 36±1° C. using circulating water warming pad and respiration rates were consistently monitored throughout the duration of the entire scan time. After obtaining the pre-injection MR images, post-injection MR imaging was performed 15 min, 30 min, 45 min, 1 h, 4 h, and 24 h after the intravenous injection of samples (20 mg of samples per kg of body weight, 80 mg of PEG-oxidized protomolecules-Fe³⁺ per kg of body weight) via the tail vein.

All of in vivo MR imaging were carried on a 7T/20 MR System (Bruker-Biospin, Fallanden, Switzerland) equipped with a 20 cm gradient set capable of supplying up to 400 mT/m in 100 μs rise-time. A quadrature volume coil (35 mm i.d.) was used for excitation and receiving the signal. MR images were obtained from each mouse liver using a fast spin-echo T1-weighted MRI sequence (TR/TE=380/7.7 ms, NEX=6, echo train length=2, 100×100 mm² in-plane resolution with a slice thickness of 1 mm and 14 slices) and T2-weighted MRI sequence (TR/TE=2000/45 ms, NEX=3, echo train length=6, 133×133 mm² in-plane resolution with a slice thickness of 1 mm and 14 slices) with respiratory gating.

Results

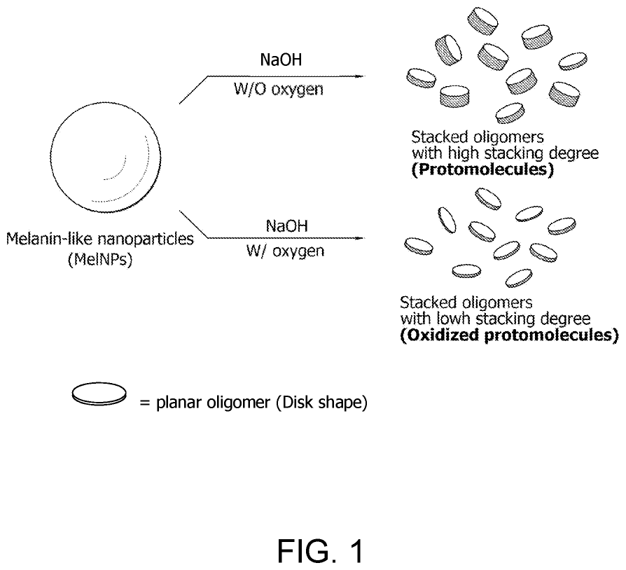

FIG. 1 (experimental scheme 1) shows the pH-controlled disassembly process for melanin-like nanoparticles (MelNPs). Under de-oxygenated condition, elevation of pH leads to disassembly of MelNPs into assembling subunits that is composed of stacked oligomers (protomolecules). In the presence of oxygen, elevation of pH results in partial chemical oxidation of protomolecules leading to de-stacking of stacked structure as well as disassembly of MelNPs into protomolecules.

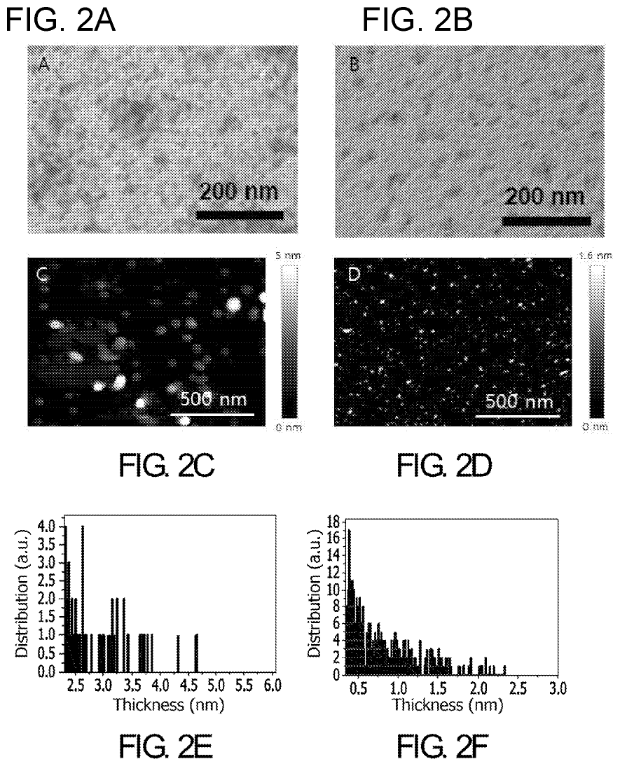

FIG. 2 provides TEM images of (A) protomolecules resulting from disassembly of MelNPs and (B) oxidized protomolecules generated by pH-controlled disassembly process in presence of oxygen. FIGS. 2(C) and 2(D) are AFM images of (A) protomolecules resulting from disassembly of MelNPs and (B) oxidized protomolecules generated by disassembly process in presence of oxygen. FIG. 2(E) shows height analysis of protomolecules and FIG. 2(F) shows oxidized protomolecules generated from disassembly of MelNPs. AFM height analysis of protomolecules and oxidized subunits was performed after they were deposited on mica substrate respectively. It showed that protomolecules and oxidized protomolecules generated by disassembly of MelNPs range from 2.2 nm to 6 nm and 0.34 nm to 2.3 nm, respectively. Given that inter sheet distance of melanin oligomers in stacked structure is about 0.34 nm, the thickness range reflects that protomolecules and oxidized protomolecules are composed of 4 to 12 oligomer sheets and 1 to 5 oligomer sheets, respectively.

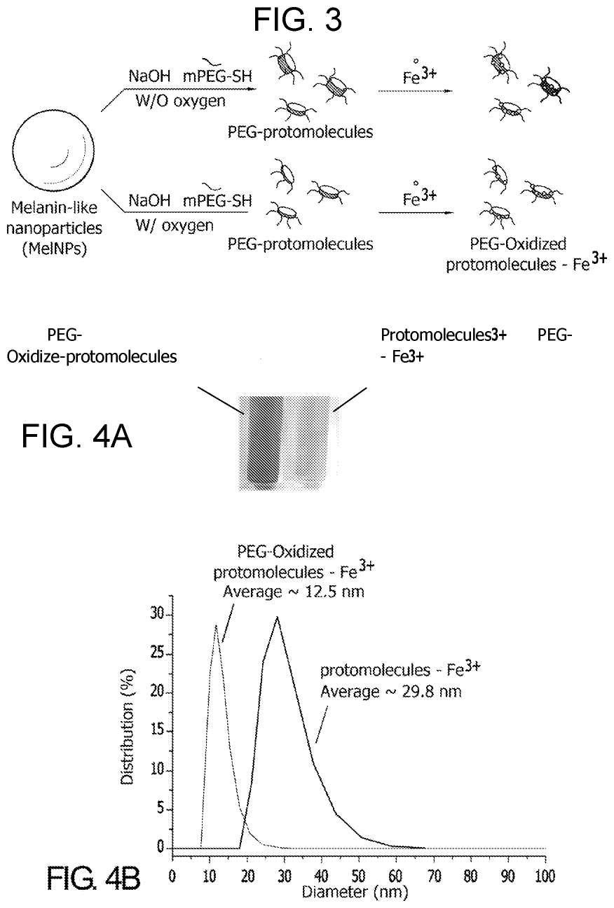

FIG. 3 (experimental scheme 2) shows magnetic resonance active protomolecules generated by disassembly of MelNPs. In the absence of oxygen, exposure to NaOH and mPEG-SH leads to disassembly of MelNPs into protomolecules and PEGylation. In the presence of oxygen, exposure to NaOH and mPEG-SH results in disassembly of MelNPs into oxidized protomolecules and PEGylation onto resulting protomolecules. After generation of protomolecules, Fe³⁺ chelation leads to T1 MRI active protomolecules.

FIG. 4(A) shows dispersion stability of PEG-protomolecules-Fe³⁺ in PBS. After chelation with Fe³⁺ ions, PEG-protomolecules-Fe³⁺ and PEG-oxidized protomolecules-Fe³⁺ showed stable dispersion behavior in PBS. FIG. 4(B) shows the hydrodynamic radius of PEG-protomolecules-Fe³⁺ and PEG-oxidized protomolecules-Fe³⁺

FIG. 5 shows T1 MR images of (A) Fe³⁺-MelNPs (˜100 nm radius)-PEG, (B) PEG-protomolecules-Fe³⁺, (C) PEG-oxidized protomolecules-Fe³⁺ with variable concentration. All images obtained under 3T MRI. Protomolecules and oxidized protomolecules shows higher contrast enhancing capability than Fe³⁺-MelNPs (˜100 nm radius)-PEG.

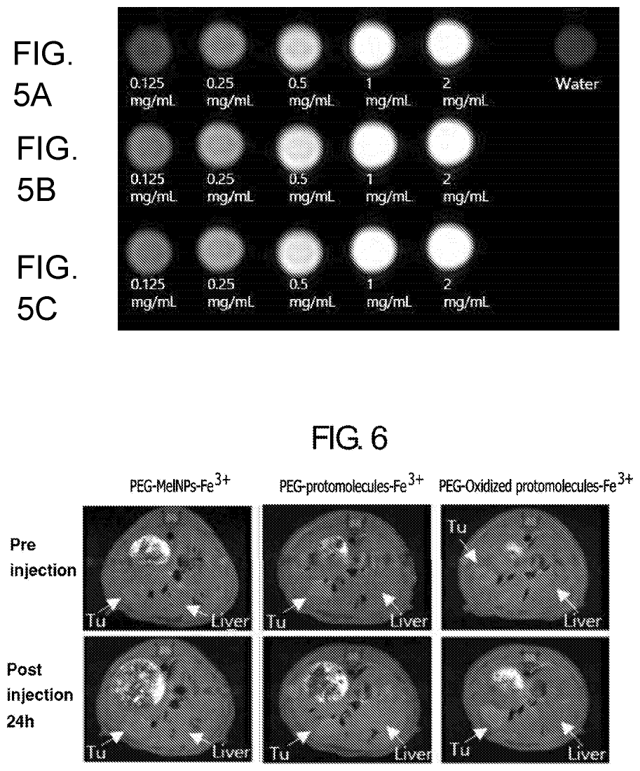

FIG. 6 shows T1 weighted MRI of mouse bearing liver tumor after injection of PEG-MelNPs-Fe³⁺, PEG-protomolecules-Fe³⁺ and PEG-oxidized protomolecules-Fe³⁺. PEG-oxidized protomolecules-Fe³⁺ showed selective contrast enhancement in tumor region 24h after injection. (Injected dosage was 20 mg of samples per kg of body weight.)

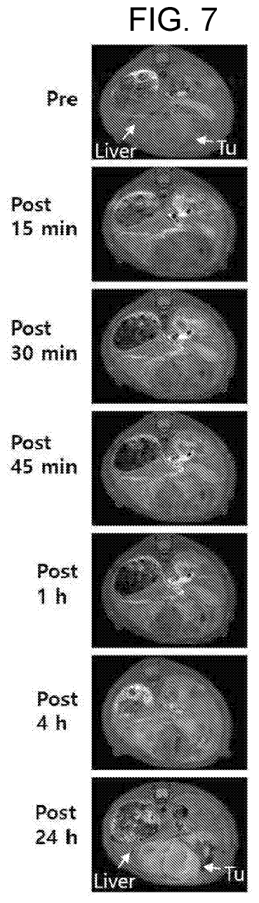

FIG. 7 shows T1 weighted MRI of mouse bearing liver tumor after injection of PEG-oxidized protomolecules-Fe³⁺. (Injected dosage was 80 mg of samples per kg of body weight.)

### Example 2

Eumelanin, the predominant type of melanin in human pigment, has been one of the most enigmatic biomacromolecules in terms of its biological function. Eumelanin is considered a beneficial biomolecule that provides protection from UV light as a result of its distinctive optical properties, broad monotonic absorption of UV-vis light and strong non-radiative relaxation of absorbed photons¹⁻³. However, eumelanin also exhibits an ability to generate reactive oxygen species (ROS) under UV irradiation⁴⁻⁶. The controversy surrounding photobiological aspects of eumelanin originates not only in the potent toxicity of photo-generated ROS but also in the relevance of these ROS to disease-related events, such as malignant melanoma. The ability of eumelanin to generate ROS under UV irradiation is likely related to malignant melanoma progression. In cutaneous pigment cells, the production of eumelanin significantly increases with the development of malignant melanoma⁷. Moreover, it has been suggested that melanin-generated ROS promotes tumorigenesis and leads to the progression of melanoma^{8, 9}.

For understanding the different photobiological aspects of eumelanin, determining the complex structure of eumelanin and revealing how eumelanin's structural organization dictates its photophysical properties is essential. Eumelanin has a hierarchically assembled particle structure composed of stacked layers of oligomers derived from two key monomers, 5,6-dihydroxyindole (DHI) and 5,6-dihydroxyindole-2-carboxylic acid (DHICA)¹⁰. Advanced spectroscopic and imaging techniques indicated the sequentially assembled structure of eumelanin, where fundamental oligomers are stacked via π-π interactions with a 3.4 Å interspace, and hydrogen bonding and hydrophobic interactions between stacked units forces the three-dimensionally aggregated particle into a scale of approximately several hundreds of nanometers¹¹⁻¹⁵. Several experimental results have provided crucial clues that help elucidate how this structural organization affects the photophysical properties of eumelanin. It appeared that the aggregation of fundamental oligomers affects not only the optical properties of eumelanin but also its photochemical reactivity upon UV irradiation, leading to ROS generation¹⁶⁻¹⁸. These results suggested that the small oligomer units of the hierarchically assembled structure are responsible for the photo-generation of ROS. Based on these observations, chemical oxidation is likely an important factor in enhancing the photochemical reactivity of eumelanin. Small angle x-ray scattering and STM studies showed that the size of the synthetic eumelanin model decreased with its chemical oxidation^{19, 20}, which suggests that chemical oxidation of eumelanin may increase its potent photochemical reactivity through the disintegration of its structure into smaller subunits.

Interestingly, the well-organized structure of natural eumelanin undergoes structural alterations in particular cases²¹. In cutaneous pigment cells, eumelanin is present with certain proteins and lipids in structures known as melanosomes²². Transmission electron microscopy studies have clearly shown the well-organized particle nature of melanosomes in normal cutaneous pigment cells²³. However, superficial spreading melanomas show a higher number of aberrant melanosome structures with an irregular internal structure, partially missing regions and disintegrated granules compared with normal pigment cells²⁴⁻²⁶. Considering the experimental finding that fundamental oligomer units of eumelanin are responsible for photochemical reactivity to generate ROS, it can be postulated that the deformation of the well-organized eumelanin structure leading to generation of smaller subunits is a critical biological process to stimulate its different photobiological function. Thus, determining the effects of the biologically relevant structural alterations of eumelanin on its photophysical properties is necessary to understanding the full spectrum of eumelanin's biological functions. However, experimental study assessing the biologically relevant structural alterations of the natural eumelanin system has been limited by the lack of a practical approach to control eumelanin's three-dimensionally assembled architecture. Two possible structural modifications can occur in eumelanin during the process of melanomagenesis: the fragmentation of its organized structure into smaller subunits with or without the oxidative destruction of the chemical structure at the level of fundamental oligomers²⁷. In these cases, either subunits or oxidized subunits disassembled from the hierarchical structure of eumelanin underlie its photophysical properties, but the well-organized hierarchical architecture of natural eumelanin has made it hard to examine the properties of its subunits. Thus, systematic investigations of the relationship between the subunit products of biologically relevant structural alterations in natural eumelanin and their photophysical and photobiological aspects have never been achieved.

The contradictory biological function of eumelanin (photoprotection vs. photosensitization) has long been a topic of debate in a wide range of disciplines such as chemistry, physics and biology. For understanding the full spectrum of eumelanin's photobiological aspect, revealing how eumelanin's complex structural organization dictates its photophysical properties is a critical step. Here, we report a practical approach to controlling the hierarchically assembled structure of natural eumelanin, which leads to disassembly of its structure into subunits and oxidized subunits respectively. Based on the well-characterized model system, it was possible to systematically determine how the photophysical properties of eumelanin are ruled by its hierarchical assembly organization. Particularly, our experiments reveal that the chemical oxidation of eumelanin's subunits, which leads to delamination of their stacked layer structure, is critical to significantly increase their photochemical reactivity to generate ROS under UV irradiation. This result provides clear experimental evidence that oxidative degradation of eumelanin, which might be induced by phagosomal enzymatic activity in the process of melanomagenesis, is responsible for triggering the negative photobiological role of eumelanin such as ROS source needed for development of malignant melanoma.

In this study, we present a novel approach to controlling the hierarchically assembled particle structure of natural eumelanin in order to disassemble eumelanin into small subunits or oxidized subunits, which are analogous to eumelanin's biologically relevant structural alterations. This practical approach involves increasing the pH level in a de-oxygenated solution to disrupt hydrogen bonds supporting the three-dimensional aggregation of eumelanin subunits. Based on this approach in a representative eumelanin model, Sepia eumelanin particles ranging from 100 nm to 300 nm could be disassembled into smaller subunits composed primarily of a stacked layer with an average stacking size of approximately 1 nm. In addition, the subunits could be further delaminated into smaller subunits by oxidative degradation under aerobic alkaline conditions. Using this well-characterized model system, it was possible to systematically determine that two key photophysical properties of natural eumelanin can be altered as a function of biologically relevant structural modifications. Particularly, our experiments revealed that the chemical oxidation of eumelanin's subunits, which leads to delamination of its stacked layer structure, is critical to significantly increase their photochemical reactivity to generate ROS under UV irradiation. Our results provide clear experimental evidence that oxidative degradation of eumelanin, which might be induced by phagosomal enzymatic activity in the process of melanomagenesis, is responsible for triggering the negative photochemical aspects and biological roles of eumelanin, such as ROS production, which is necessary for the progression of malignant melanoma.

Preparation of Sepia Eumelanin.

Sepia was extracted using a syringe from a freshly dissected ink sac and collected by centrifugation (18000 rpm) as described in a previous study.³³ Ink sacs were obtained by the dissection of Korean cuttlefish. In order to examine the disassembly and oxidation process of Sepia eumelanin, Sepia eumelanin was purified by more than 10 times of washing processes of centrifugation and re-dispersion in water.

Synthesis of MelNPs.

MelNPs were synthesized by a slight modification of previously reported methods²⁹. A description of the synthetic conditions is provided as follows; 180 mg of dopamine hydrochloride (Aldrich Chemical) was dissolved in 45 mL of deionized water. 670 μL of NaOH (1N) solution was added to a solution of dopamine hydrochloride under vigorous stirring at 50° C. After 5 h, MelNPs were isolated and purified by several times of centrifugation/re-dispersion in deionized water (4000 rpm for 10 min).

Disassembly and Simultaneous Disassembly/Oxidation Process for Sepia Eumelanin.

For examination of disassembly aspect of Sepia eumelanin, solution of Sepia eumelanin (1 mg/mL) was added into buffer solution (pH 9.5) under N₂ purging. All solution was pre-purged by N₂ for 20 min to eliminate dissolved O₂ in solution. After 10 min, partially disassembled Sepia particles can be separated from released subunit by centrifugation (13500 rpm, 10 min) and re-dispersion in water. For examination of morphological change of Sepia under pH-controlled disassembly, partially disassembled Sepia was directly transferred to a carbon-coated TEM grid for TEM investigation. For fully disassembled Sepia, 3.5 mL of NaOH solution (1 M) was added into 1 mL of Sepia solution (1 mg/mL) under N₂ purging. All solution was pre-purged by N₂ for 20 min to eliminate dissolved O₂ from the solution. After 12 h, 5 mL of KH₂PO₄ solution (0.1 M) was added into solution of fully disassemble Sepia to neutralize the solution pH and prevent additional pH-induced oxidation of subunits under ambient environment. Because subunits generated from Sepia eumelanin shows aggregation behavior after 1 week, freshly generated subunits was used to examine their optical properties and photochemical reactivity. Oxidation process of Sepia eumelanin was progressed for 5 days with same manner as described above excepting N₂ purging. Generated oxidized subunits shows very stable dispersion stability in the neutralized buffer solution for more than 1 month.

Characterization of Subunits and Oxidized Subunits from Sepia Eumelanin.

For examination of morphological change of Sepia under pH-controlled disassembly and oxidation process, subunits disassembled from Sepia and oxidized subunits were characterized by TEM. After the disassembly and simultaneous disassembly/oxidation process, absorbance of equivalent concentration of subunits and oxidized subunits were characterized via UV-vis spectroscopy (SINCO S-3100). Emission spectra of each sample were measured by a Jasco FP-6500 spectrofluorometer at the excitation wavelengths of 314 nm. Excitation spectra for each emission maximum wavelength of the emission spectra were obtained under the same experimental setup. ¹³C NMR spectra of oxidized subunits compared to non-oxidized subunits and their parental Sepia were collected on a Bruker 400 MHz solid/micro-imaging high resolution NMR spectrometer.

For AFM analysis, the subunits and oxidized subunits generated from Sepia eumelanin were dialyzed respectively using a dialysis kit (Thermo Scientific Slide-A-Lyzer Dialysis, 2 K MWCO) for 1 day. This was performed in order to eliminate salt, and to rule out the oligomeric species (MW<2000) from the whole subunits mixture. After the dialysis, the diluted subunit solutions were spin-coated on a mica substrate (4000 rpm, 30 sec) for AFM investigation. The AFM experiments were performed using a Nanoscope IIIc microscope (Veeco Instruments, Santa Barbara, Calif.) in air at ambient temperature. All images were obtained on tapping mode using non-contact mode tips from BRUKER with a spring constant of 40 Nm-1 and a tip radius of <12 nm. Oligomeric unit fraction (MW<2000) can be separated from the whole mixture of subunits or oxidized subunits through dialysis and they are concentrated via evaporation of water. The concentrated oligomeric unit fraction (MW<2000) were characterized via UV-vis spectroscopy and spectrofluorometer as method mentioned above.

Photogeneration of Reactive Oxygen Species (ROS) by Sepia Eumelanin in Response to the Structural Alterations.

The generation of superoxide radical was monitored by the nitro blue tetrazolium (NBT) method, which measured the changes of diformazan absorption at 560 nm, resulting from the reduction of NBT with superoxide radical during the photoirradiation.^{28, 29}

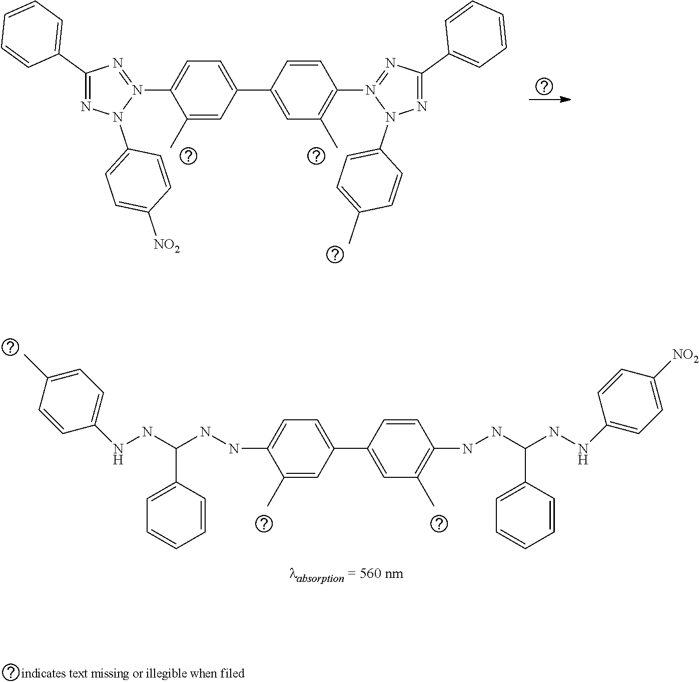

200 μL of freshly prepared NBT solution (1 mM) was dissolved in 2 mL of phosphate buffer solution (1 M) in a quartz cuvette, and an appropriate amount of Sepia samples (Sepia particles, subunits, oxidized subunits before and after dialysis) was added to the cuvette, individually. The total amount of subunits was varied as 30 μg excepting for oxidized subunits after dialysis. After mixing the solution, the quartz cuvette was exposed to the irradiation of a xenon lamp (100 W) at a distance of 10 cm. Absorption from diformazan at 560 nm was obtained as a function of time during the irradiation.

For monitoring the generation of the hydroxyl radical, coumarin-3-carboxylic acid (CCA) was used owing to the characteristic emission at 446 nm when it reacts with hydroxyl radical to produce 7-hydroxycoumarin-3-carboxylic acid (7-OHCCA).^{30, 31}

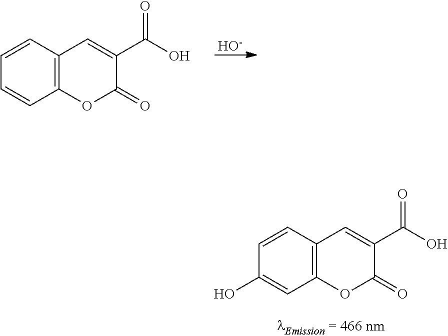

CCA was dissolved in deionized water by adding 20 μL of ammonia solution (28-30%) and its concentration was adjusted as 5 mM. 200 μL of the freshly prepared CCA solution was added into 2 mL of phosphate buffer solution (1 M) in a quartz cuvette, and an appropriate amount of Sepia samples was added to the cuvette, individually. The total amount of subunits was 30 μg excepting for oxidized subunits after dialysis. After mixing the solution, the quartz cuvette was exposed to the irradiation of a xenon lamp (100 W) at a distance of 10 cm. Emission spectra from 7-OHCCA (λex=380 nm) were obtained as a function of time during the irradiation.

Results and Discussion

pH-Controlled Disassembly and Simultaneous Disassembly/Oxidation Process for Sepia Eumelanin.

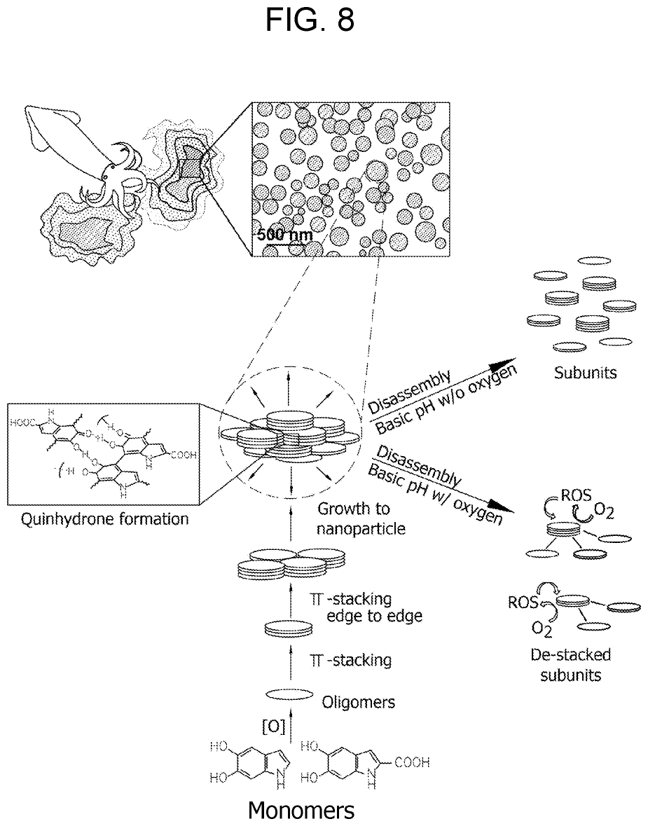

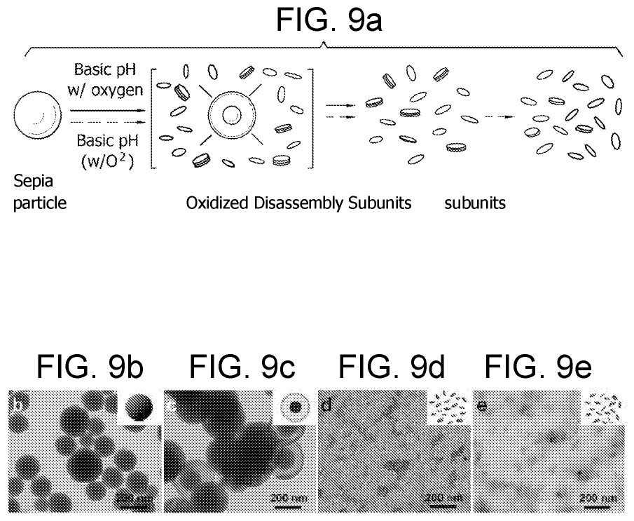

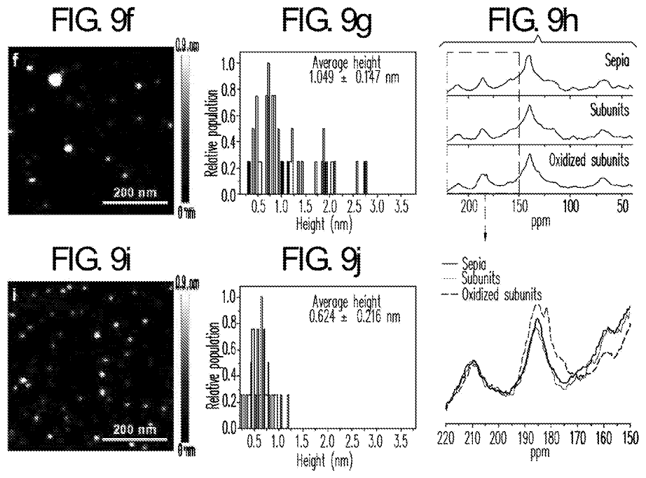

Sepia eumelanin particles were utilized as a eumelanin model with a hierarchically assembled particle structure. Sepia, a well-characterized natural eumelanin, has been used as eumelanin model to study the physico-chemical properties of eumelanin^{16-18, 32} due to its high purity and relatively simple purification procedure. The hierarchical structure of Sepia eumelanin, which comprises an aggregate of stacked-oligomers, has been well characterized by SEM³³, AFM¹⁵ and wide-angle X-ray diffraction measurement¹². As shown in FIGS. 8 and 9-b, Sepia eumelanin extracted from the ink sack of cuttlefish showed the characteristics of well-organized particles with a broad size distribution ranging from 100 to 300 nm. Because the aggregated particle nature of eumelanin is finally forced by edge-to-edge joining of the stacked oligomers through hydrogen-bonding in a quinhydrone formation (FIG. 8), the disruption of hydrogen bonding by increasing pH of the solvent (water) could induce the disassembly of Sepia eumelanin particles in a manner reminiscent of the denaturation of the protein's secondary structure. In addition to increasing solvent pH, the de-oxygenation of the solvent is important for preventing chemical oxidation of the generated subunits. The primary dihydroquinone moiety of eumelanin monomers can be easily oxidized by dissolved oxygen in alkaline pH conditions, and oxidizing the dihydroquinone moiety results in the generation of reactive oxygen species (ROS) via a chain autoxidation process between the oxidized form of dihydroquinone and dissolved oxygen³⁴. Accordingly, Sepia subunits generated by the disassembly process in the presence of oxygen can undergo further chemical oxidation by the generated ROS, such as superoxide radical and hydrogen peroxide. Therefore, the Sepia solution was purged by N₂ during the disassembly process in order to prevent the oxidation of the generated subunits. The morphological changes of Sepia eumelanin were monitored by TEM after exposure to de-oxygenated basic pH solution (pH 9.5) followed by centrifugation to separate the released subunits (FIG. 9-c). Several Sepia particles displayed a swollen yolk-shell structure because of the released subunits from the inside of the particles due to the disassembly process. The disassembly of Sepia particles leading to the release of its subunits is increased in higher pH conditions with equivalent exposure time (FIG. 15), indicating that the disassembly process more effectively progresses under higher pH levels via enhanced disruption of hydrogen bonding in the hierarchical structure of Sepia. Sepia eumelanin particles could be completely transformed to small-sized subunits after exposure to de-oxygenated NaOH solution (0.5 M) for 12 h (FIG. 9-d); these smaller subunits could be stored stably in neutralized buffer solution after adding potassium dihydrogen phosphate solution. Because they shows aggregation behavior after 1 week, freshly generated subunits were utilized for characterization of their photophysical properties.

The oxidation of subunits disassembled from Sepia particles could be induced by allowing dissolved oxygen in NaOH solution during the disassembly process. As described above, the autoxidation of dihydroquinone moiety in oxygen-dissolved alkaline solutions results in the generation of ROS, such as superoxide radical and hydrogen peroxide.³⁴ Therefore, increasing solvent pH in the presence of dissolved oxygen not only caused the disassembly of Sepia eumelanin particles into subunits but also led to their chemical oxidation by generated ROS, which is very similar to the oxidative bleaching process for eumelanin via alkaline peroxide solution treatment²⁰. Chemically oxidized subunits were obtained by exposure to NaOH solution (0.5 M) containing dissolved oxygen for 5 days and stored in neutralized solution by adding potassium dihydrogen phosphate solution (FIG. 9-e). They showed very stable dispersion stability in the neutralized buffer solution for more than 1 month.

Characterization of Subunit Products Resulting from the Disassembly and Simultaneous Disassembly/Oxidation of Sepia Eumelanin.

Previous matrix-assisted laser desorption/ionization (MALDI) mass spectrometry studies showed that mass of Sepia protomolecules was less than 1200, and high molecular weight of polymers was not observed³⁷ Scanning tunneling microscopic studies showed that lateral size of eumelanin protomolecules is approximately 2 nm.^{21, 38} These results suggests that oligomer units composed of covalently connected MI or DHICA would be fundamental units of eumelanin. Many studies based on advanced imaging techniques^{12, 21, 38, 39} and theoretical calculation⁴⁰⁻⁴² have elucidated Sepia eumelanin as hierarchical assembly system where the fundamental oligomer units are stacked through π-π interaction and the stacked layers of oligomeric units (protomolecules) form a three-dimensionally aggregated particle structure driven by non-covalent interactions such as hydrophobic interaction and hydrogen-bonding (FIG. 8). Because pH-controlled disassembly process results in complete disintegration of three-dimensionally aggregated particle structure of Sepia into small subunits, considering the hierarchical assembly picture of eumelanin, fundamental oligomer units, stacked oligomers and cluster of the stacked oligomers would be possible constituents of the resulting subunits.

For the structural characterization of Sepia subunits, the subunits generated from pH-controlled disassembly process were divided in terms of molecular weight. Through dialysis with a molecular weight cutoff at 2000, subunit fraction with molecular weight less than 2000 was separated from the entire subunits. Given the molecular weight range of Sepia fundamental oligomer species observed by the previous mass spectrometry studies, the oligomer units were separated from the entire subunits after dialysis. By comparing UV-vis absorption spectra before and after the dialysis process, the approximate proportion of subunit fraction with MW less than 2000 relative to the entire Sepia subunit mixture could be predicted. As shown in FIG. 11-b, slightly decreased absorption contributing to the UV and blue regions was observed after the dialysis process. Even though the lack of a clear molar absorption coefficient for both subunit fractions makes it difficult to predict precise proportions of each subunit fraction by comparing UV-vis absorption spectra before and after dialysis, very slight decrease in UV-vis absorption after the dialysis process indicates that the subunit fraction with molecular weight less than 2000 is very small in the whole subunits mixture, while another subunit fraction with molecular weight more than 2000 is main product resulting from pH-controlled disassembly of Sepia eumelanin.

Thickness of the main subunit fraction (MW>2000) as a result of disassembly of Sepia was analyzed by atomic force microscope (AFM) after depositing the subunit fraction on mica substrate through dropping diluted subunit solution on a spinning mica substrate. It is hard to estimate lateral size of the subunits because the aggregation of the subunits could occur in the x-y direction during the sample preparation process. However, height analysis of subunits on the mica substrate provides information about the thickness of subunits.²¹ The average height of the subunit fraction (MW>2000) appeared to be 1.0±0.1 nm (FIG. 9-f and g). Given the previous finding that inter-sheet distance in stacked oligomers of Sepia eumelanin is 0.34 nm,¹² the average height indicates that the subunit fraction (MW>2000) are multi-layered oligomers with 3-4 sheets. On the other hand, small portion of subunit fraction (MW<2000) would be composed of single oligomers and thin-layered oligomers small enough to pass through dialysis membrane.

Thickness of the main subunit fraction is well matched with the height dimension of eumelanin protomolecules that was previously observed by STM. Synthetic melanin deposited on graphite crystals showed height of 1-1.5 nm, which has been suggested as eumelanin protomolecules of stacked oligomers.^{21, 38} Therefore, it is reasonable to expect that Sepia eumelanin protomolecules of multi-layered oligomers with 3-4 sheets are major products generated from pH-controlled disassembly of Sepia. In the light of hierarchical structural picture of Sepia eumelanin, generation of Sepia protomolecules reflects that non-covalent interaction forcing eumelanin protomolecules aggregated would be significantly attenuated under de-oxygenated alkaline condition, which reads to complete disintegration of Sepia eumelanin. As indicated in FIG. 8, hydrogen bonding between the protomolecules, giving rise to the polyquinhydrone complex,^{43, 44} has been regarded as an important secondary interaction responsible for edge-to-edge joining of eumelanin protomolecules.^{19, 45} Because redox equilibrium of eumelanin can be changed as a function of pH,⁴⁶ disruption of hydrogen bonding would be a possible factor to induce disassembly of Sepia eumelanin under de-oxygenated alkaline condition. Increase in pH level would shift its equilibrium from the polyquinhydrone complex to polyquinone, which is possibly related to disruption of hydrogen bonding between the protomolecules.

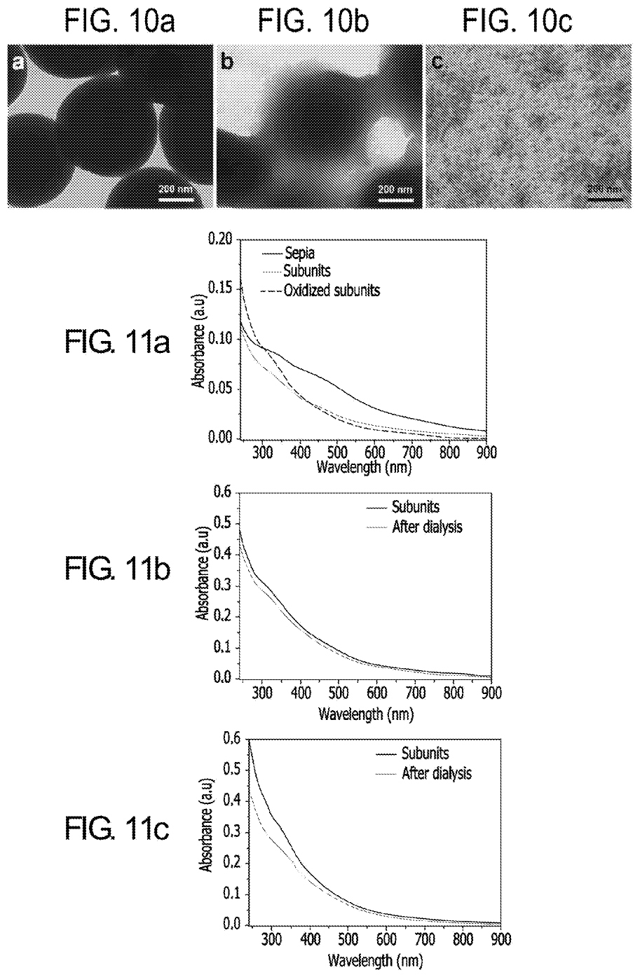

Since the Sepia eumelanin contains proteins with weight portion about 5-10%,⁴⁷ proteins could be another possible factor to induce pH-controlled disassembly of Sepia. However, it has been shown that selective digestion of the protein surrounding natural eumelanin by using proteolytic enzyme doesn't affect its particle structure.^{48, 49} This result indicates that protein portion surrounding eumelanin particle is not critical factor to retain three-dimensionally aggregated particle shape of eumelanin. For more understanding of pH-induced disassembly phenomena with relation to effect of protein, pH-controlled disassembly of synthetic melanin-like nanoparticles (MelNPs) was examined. MelNPs that we previously developed are synthetic melanin model generated from spontaneous oxidation of dopamine.²⁹ Without any involvement of biological molecules confining particle structure, simple autoxidation of dopamine in water resulted in particular shape of melanin. Recent structural studies suggested that structure of products generated from autoxidation of dopamine is very similar with hierarchical assembly of natural eumelanin.^{45, 50} Because there is no involvement of biological molecule in synthetic model, MelNPs are proper model to examine pH-induced disassembly of Sepia eumelanin with relation to involvement of biological molecules such as protein. As shown in FIG. 10, well-organized particle structure of MelNPs was also disassembled into small subunits after exposure to de-oxygenated basic solution. Disassembly of synthetic melanin model in response to increased pH indicates that biological molecules deposited in Sepia eumelanin are not critical factor with relation to the pH-controlled disassembly process.

Subunits generated by simultaneous pH-controlled disassembly/oxidation of Sepia were characterized in the same manner as the non-oxidized subunits. Through the dialysis process, the subunits fraction (MW<2000) was separated from the entire oxidized subunits. The elimination of the subunits fraction (MW<2000) also resulted in decreased UV-vis absorption spectrum (FIG. 11-c). In this case, a much larger proportion of absorption in the UV region was decreased compared with that of non-oxidized subunits as a result of eliminating the subunits fraction. An increased amount of small subunit, capable of passing through dialysis membrane, is a possible explanation of this result. A previous NMR study of the oxidation of Sepia eumelanin indicated that chemical oxidation causes the partial degradation of eumelanin oligomers, generating a pyrrole carboxylic acid group in the oligomer structure.⁵¹ Therefore, the attenuation of π-π interactions between stacked oligomers induced by the oxidative partial degradation of fundamental oligomers may result in the delamination of stacked layers. This expectation could be supported by experimental results showing decreased size of synthetic eumelanin model in response to chemical oxidation.^{19, 21} Chemical oxidation of the eumelanin model with an alkali peroxide solution has been demonstrated to decrease its particle size.^{19, 21} In a similar way, stacked oligomer fractions generated from a pH-controlled disassembly/oxidation process could be delaminated through oxidative partial destruction by generated ROS, as consequently, the number of small subunits would be increased. This expectation could be confirmed by the size analysis of oxidized subunits by AFM measurement. The average thickness of oxidized subunit fraction (MW>2000) decreased to 0.6±0.2 nm compared with the corresponding non-oxidized subunit fraction (MW>2000) with a size of 1.0±0.1 nm (FIG. 9-f, g, i and j). The decreased thickness of the subunit fraction (MW>2000) indicates that they undergo the delamination under pH-controlled oxidation processes, which leads to an increased proportion of single and thin-layered oligomers species. A CP-MAS ¹³C solid-state NMR study provided further insight into the effects of the pH-controlled oxidation process on structural modifications to Sepia eumelanin subunits. As shown in FIG. 9-h, characteristic spectra ranging from 180 to 185 ppm, which correspond to the carbonyl resonances of the generated pyrrole carboxylic acid, was clearly observed in oxidized subunits, while parental Sepia and non-oxidized subunits did not show any signal in that range. This oxidative partial destruction of subunits would be a crucial factor in promoting oxidation-induced delamination of the Sepia subunit structure via the attenuation of π-π interactions between stacked oligomer units.

Absorption Spectra of Sepia Subunits and Oxidized Subunits.

It is found that monotonically increasing broad UV-vis absorption moving toward the UV region, key optical property of eumelanin related to its photoprotection function,^{52, 53} was changed as a function of structural modification. Compared with the absorption pattern of parental Sepia, the wide range of visible absorbance was decreased through the disassembly and simultaneous disassembly/oxidation processes (FIG. 11-a).

De-aggregation of stacked oligomers is a possible factor to contribute to disassembly-dependent absorption spectrum of Sepia eumelanin. Several theoretical calculations and pulse radiolytic studies have suggested that the optical properties of eumelanin are only governed by the degree of π electron delocalization within oligomeric units that are fundamental constituents of eumelanin.^{52, 54, 55} Based on this model, the broad absorption of eumelanin is interpreted as an ensemble of chemically distinct species with various absorption bands, where the oligomerization of monomeric units with various conformations, configurations and redox states causes their absorption bands to be redshifted, broadening and diverse. However, recent theoretical and experimental efforts have further illuminated this picture to the secondary structural level, suggesting that non-covalent interactions, such as stacking and aggregation, also play an important role in the optical properties of eumelanin. ^{40, 42, 56, 57} This additional information indicates that close proximity of subunits leads to their electronic coupling, which allows HOMO-LUMO gap energy of fundamental units to further shift to lower energy. Because hydrogen-bonding would responsible for linking stacked oligomers to be aggregated, the disruption of hydrogen bonding may significantly decrease electronic coupling between stacked subunits. Therefore, the proportion of optical density, especially around visible and near IR, arising from the aggregation of stacked oligomers could be reduced via the disassembly of aggregated particle structures.

Oxidative degradation of subunits resulted in a further decrease in their optical density in addition to the disassembly of Sepia particles. This result can be explained by two distinct effects. First, oxidative partial degradation of Sepia eumelanin yields pyrrole carboxylic groups within their oligomers,⁵¹ and thus, the π electron de-localization system of oligomer units would be shortened. As a result, the generation of pyrrole carboxylic acid groups in fundamental oligomers could lead to blue-shifting of the HOMO-LUMO gap of oligomer units. Another possible cause of the decreased absorption is the de-stacking of the stacked layer structure induced by oxidative partial degradation of subunits. In this case, decreased electronic coupling between oligomers within stacked species would further contribute to decreased absorption around the visible and near IR region.

It is noteworthy that involvement of protein in UV-vis absorption spectrum of Sepia seems negligible. In the previous study about UV-vis absorption spectra of size-selected Sepia eumelanin, it was found that clear absorption peak of protein is observed around 270 nm.¹⁸ Absorption peak originated from protein is more clearly observed in size-selected Sepia fraction with molecular weight less than 1000, which indicates that majority of protein fraction in Sepia have molecular weight less than 1000. In the other hand, any appreciable peak near 270 nm in the monotonically increasing absorption of Sepia was not observed in this study. Furthermore, both size-selected subunit fraction disassembled from Sepia particles didn't show any absorption peak around 270 nm. This observation reflects that most of protein portion in Sepia eumelanin particle would be removed and thus, its absorption contribution is negligible. It is expected that more than 10 times of sequential centrifugation/re-dispersion of Sepia particle in water would result in effective removal of protein portion in Sepia particles.

Fluorescence Emission Spectra of Sepia Subunit and Oxidized Subunits.

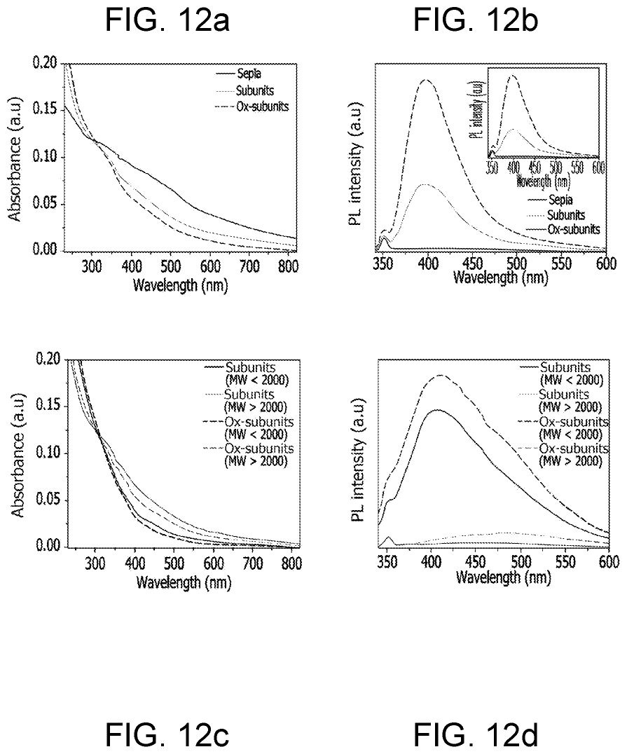

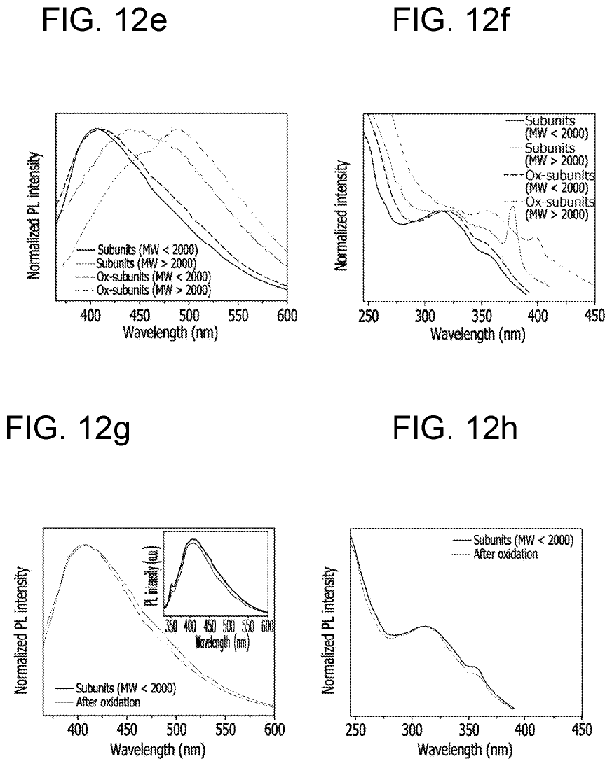

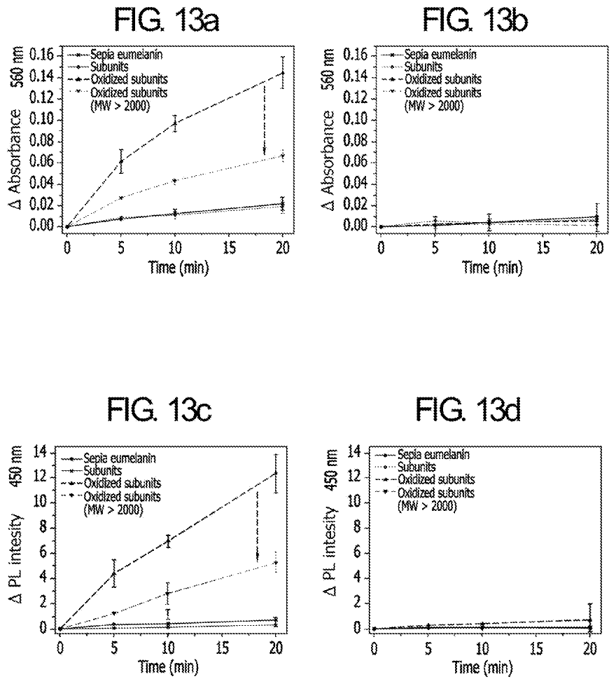

Considering that UV light exposure is highly responsible for skin damage and skin cancer, efficient UV dissipation through strong non-radiative relaxation process is another key optical property of eumelanin with relation to its photo-protective functionality. ³ Therefore, it has been well known that eumelanin exhibits a very low intensity of fluorescence emission when it is excited by UV light. However, the extremely low fluorescence emission intensity of Sepia eumelanin increase with its structural disassembly into small units and further oxidization. At the equivalent weight concentration of each sample, subunits disassembled from Sepia particles yielded enhanced fluorescence emission between 350 and 550 nm with excitation at 314 nm (UV-B) compared with parental Sepia (FIG. 12-b). In addition, oxidized subunits exhibited further increased fluorescence intensity compared with non-oxidized subunits. In order to minimize problematic phenomena, such as the attenuation of the excitation beam and re-absorption of emitting light, the absorbance of each sample at excitation wavelength was tuned to be equivalent and their concentration was highly diluted (FIG. 12-a). In this case, they showed very similar structure-dependent fluorescence emission (FIG. 12-b). This result indicates that both de-aggregation of Sepia protomolecules and oxidation of the protomolecules are closely related to decrease in strong non-radiative relation process.

To better understand the fluorescence emission of Sepia eumelanin as a function of its structural alteration, emission, excitation and absorption spectra of size-selected subunits were compared. As described above, the subunits products generated from the pH-controlled disassembly process or simultaneous disassembly/oxidation process could be separated in terms of molecular weight through the dialysis process. It is noteworthy that the subunit fraction (MW<2000) showed a distinguishable absorption peak at 314 nm. When the subunits fraction (MW<2000) are excited at 314 nm, they showed broad emission peak in the visible (FIG. 12-d). The excitation spectrum corrected at the wavelength of emission maximum exhibited a characteristic peak around 314 nm (FIG. 12-f). The characteristic absorption band of the subunits fraction near 314 nm would be attributed to single fundamental oligomer units. It has been shown that S₀-S₁ transition of the fundamental oligomeric components of eumelanin makes their absorption band observed around 314 nm.^{56, 58} Therefore, it is expected that the single oligomeric units are main composition responsible for the intense fluorescence emission of the subunits fraction (MW<2000) when they are excited at 314 nm.

In the other hand, subunits fraction composed of stacked oligomers (MW>2000), they showed weaker fluorescence emission intensity than oligomeric units of another subunits fraction (MW<2000) (FIG. 12-d). In the light of structural difference between two subunit fractions, stacking structure of the subunit fraction (MW>2000) is a possible structural factor to influence on its red-shifted and weak fluorescent emission. As described above, theoretical calculation studies predicted that close proximity of the fundamental oligomeric units results in electronic coupling between them and leads to red-shifting of their HOMO-LUMO gap^{40, 57}. Comparison of the absorption spectra between two subunits fractions provide insights into the electronic coupling between fundamental oligomer species confined in stacked structure. As shown in FIG. 12-c, the stacked oligomeric unit fraction showed more absorption contribution in the visible and near IR regions than the subunit fraction with molecular weight less than 2000. Electronic coupling between fundamental oligomeric units in stacked structure is also found in the emission spectra. Emission spectrum of the stacked oligomeric unit fraction shows broader and red-shifted emission peak than the unstacked oligomeric units in subunit fraction (MW<2000) (FIG. 12-e). In addition, corresponding excitation spectrum of the stacked oligomeric unit fraction also exhibited red-shifting of characteristic peak near 314 nm (FIG. 12-f). The electronic coupling between stacked oligomers may be attributed to the stacking-mediated fluorescence quenching. Broad and redshifted emission is characteristic of self-assembled system of aromatic chromophores. When one of chromophores is electronically excited in the assembled system, the molecules forms excimer complex in excited state and they exhibits broad and redshifted emission. ^{59, 60} As a similar manner, stacked layers of Sepia oligomers would undergo electronic coupling in excited state and new energy states generated by the excimer formation may be involved in strong non-relaxation process of the whole stacked oligomer system. However, exact relaxation process associated with stacking structure is unclear now. More detailed study on the stacking-mediated fluorescence quenching mechanism of Sepia subunits is on the way

The stacking-mediated fluorescence quenching behavior of Sepia subunits could be linked to the oxidation-induced fluorescence enhancement of Sepia eumelanin, because subunits composed of stacked oligomer units are prone to be de-stacked as a result of pH-controlled oxidation. Therefore, we predicted that the decreased stacking size of the stacked oligomeric fraction (MW>2000) induced by oxidation would make absorption contribution in visible region decreased and strong non-radiative relation process attenuated. As expected, normalized absorption spectrum of the oxidized subunit fraction composed of stacked layers (MW>2000) showed decreased absorption contribution in the visible region compared with the corresponding non-oxidized subunit fraction (FIG. 12-c). In addition, the oxidized subunit fraction showed enhanced fluorescence intensity in comparison to the corresponding non-oxidized subunit fractions. These results are consistent with the stacking effect expected in the comparison between the non-oxidized unstacked and stacked oligomer fractions.

The oxidized subunit fraction with molecular weight less than 2000 also showed increased fluorescence intensity compared with the corresponding non-oxidized subunit fraction (MW<2000). The delamination of stacked oligomers induced by oxidation may result in the generation of highly florescent single and thin-layered oligomers capable of passing through a dialysis membrane. Accordingly, the newly generated small subunits would contribute to the enhanced fluorescence intensity of the oxidized oligomeric subunit fraction (MW<2000). For understanding relatively high fluorescence intensity of oxidized subunit fraction (MW<2000) compare to non-oxidized subunit fraction (MW<2000), isolated subunits fraction (MW<2000) was selectively oxidized by pH-controlled oxidation process and their fluorescence emission behavior was compared with the oxidized subunit fraction (MW<2000) generated from simultaneous pH-controlled disassembly/oxidation process. Considering the fact that fluorescence emission of subunit fraction (MW<2000) with excitation at 314 nm is mainly generated from fundamental oligomeric units, selective oxidation of the subunit fraction (MW<2000) offers insight into understanding fluorescence emission behavior of the fundamental oligomeric units in response to ROS-mediated oxidation. As shown in FIG. 12-g, the isolated subunit fraction (MW<2000) showed slightly decreased fluorescence emission intensity after pH-controlled oxidation. In addition, the emission contribution in lower energy region was decreased after oxidation (FIG. 12-g) and its corresponding excitation spectrum exhibiting a characteristic peak at 314 nm showed slightly blue-shifted behavior as a function of oxidation (FIG. 12-e). On the other hand, fluorescence emission spectra of oxidized subunits fraction (MW<2000) as a result of simultaneous disassembly/oxidation showed red-shifted emission spectra compared with the corresponding size-selected non-oxidized subunits (FIG. 12-e). These results indicate that chemical oxidation of eumelanin oligomeric units doesn't make their fluorescence emission intensity enhanced. The slightly decreased fluorescence emission is possibly originated from their oxidative partial degradation accompanying change of their π conjugation system. Based on this result, we concluded that single or thin-layered oligomers generated by the oxidation-induced delamination of stacked oligomeric units contributes to enhancement of fluorescence intensity of two subunits fractions.

Photochemical Reactivity of Sepia Subunits and Oxidized Subunits.

Finally, the photochemical reactivity of Sepia eumelanin models to generate ROS in aerobic conditions was examined to explore the relationship between its hierarchical structure, optical properties and photobiological aspect. Major ROS products, superoxide and hydroxyl radicals, generated from photo-irradiated Sepia eumelanin in aerobic water solution were monitored as a function of eumelanin's structural alteration. The photo-generated radical species from the Sepia eumelanin models were observed by ROS probes, such as nitro blue tetrazolium (NBT)^{28, 29} and coumarin-3-carboxylic acid (CCA)^{30, 31}. The methods are based on the evolution of absorption from a reduced form of NBT by the superoxide radical and emission from the hydroxylated form of CCA, respectively. As shown in FIG. 12-a and c, subunits disassembled from parental Sepia showed negligible signal enhancement of the superoxide and hydroxyl radicals compared with parental Sepia particles during photo-irradiation. However, oxidized subunits showed enhanced photochemical reactivity to generate ROS. The capability to generate ROS for oxidized subunits was decreased to be comparable to the level of the parental Sepia particle and non-oxidized subunits in the absence of light irradiation (FIG. 12-b and d). This indicates that the detected ROS were not generated by chemical reaction between oxidized subunits and dissolved oxygen but were the result of the photochemical reaction of oxidized subunits with oxygen molecules.

Enhanced photochemical reactivity of oxidized subunits compared with their parental Sepia particle and non-oxidized subunits is likely associated with several structural factors. The non-radiative relaxation pathway of photo-induced electrons in eumelanin would compete with the process to activate oxygen and generate ROS. Thus, it can be expected that relatively high fluorescence subunit species are more photo-reactive than low fluorescent stacked oligomers. Therefore, newly generated fluorescent species as a result of pH-controlled oxidation of Sepia subunits would increase the photochemical reactivity of oxidized subunits. As indicated in FIG. 12-a and c, oxidized subunits still showed appreciable ROS generating capability after removal of the subunit fraction (MW<2000), which reflects that newly generated florescent subunit species including stacked oligomers of decreased stacking size in the subunit fraction (MW>2000), as well as single and very thin-layered oligomers in the other subunit fraction (MW<2000), are responsible for the enhanced photochemical reactivity of oxidized subunits. In addition, eumelanin can serve not only as a photosensitizer producing ROS but also as an antioxidant capable of quenching generated ROS^{4, 50-52}. Accordingly, the quantity of photo-generated ROS determined by chemical probes is a result of the combination of two distinct functions. Because the oxidation of Sepia subunits leads to partial degradation of the dihydroquinone moiety responsible for ROS quenching capability, it is reasonable to expect that a decreased ROS quenching capability of oxidized subunits along with their increased photochemical reactivity would result in an increased amount of photo-generated ROS detected by chemical probes. In the case of the non-oxidized subunit fraction, a very small portion of single oligomer species (MW<2000) may generate ROS when irradiated, but the amount of photo-reactive species would not be enough to overwhelm the ROS quenching capability of the majority of stacked subunits, which possibly makes their photochemical reactivity determined by chemical probe negligible. From this structural implication in the photo-generation of ROS, the parental Sepia particle has a great advantage in retaining its photochemical stability because even a small portion of photo-reactive single oligomer species is confined by the three-dimensionally aggregated particle structure. This hierarchically assembled structure of Sepia may be an architectural strategy utilized to retain its photo-protective function while deactivating its photochemical reactivity.

Proposed Mechanism of the Janus Behavior of Eumelanin.

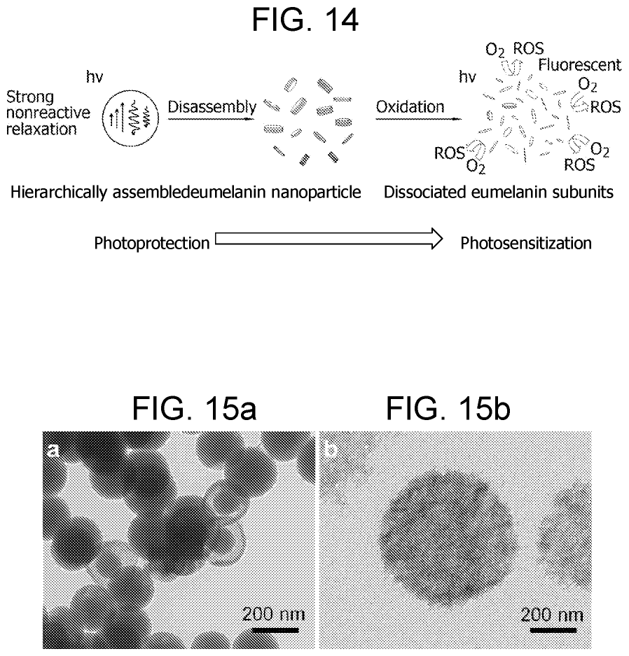

Substantial and systematic alterations of photo-physical properties of Sepia eumelanin in response to its biologically relevant structural alterations were clearly observed using a well-organized model system, the pH-controlled disassembly and simultaneous disassembly/oxidation process. This result suggests that the contradictory biological functions of eumelanin (photoprotection vs. photosensitization) are closely related to the oxidation of their subunits. The proposed structure-property-function relationship of eumelanin, emphasizing the importance of the oxidation of eumelanin's subunit structure, is schematically summarized in FIG. 14.

Given that the fluorescent single oligomer species is highly responsible for the photo-reactivity of eumelanin, we conclude that the hierarchically assembled structure of eumelanin, including the aggregation of stacked oligomer units and the stacking structure of fundamental oligomeric units, may be a critical structural strategy that eumelanin adopts to deactivate the photochemical reactivity of its oligomeric subunit species while providing photoprotective functions. According to this hypothesis, eumelanin will offer efficient photo-protective function against UV light through broad monotonic absorption and strong non-radiative relaxation processes. Even if the aggregated particle structure of eumelanin is disrupted by particular biological factors and disintegrated into subunits, the majority of stacked subunits will still serve as photo-protective biomolecules because their stacking structure prefers non-radiative relaxation processes when irradiated. However, structural alteration accompanying the de-stacking of stacked oligomers by overloaded oxidative stress in a bio-system will significantly alter the photobiological aspects of eumelanin and cause it to play a different biological role. This perspective pointing to the structure-biological function relationship of eumelanin provides insight into predicting various photobiological aspects of eumelanin with relation to melanomagenesis. Abnormal structural alteration of melanosomes exhibiting disintegration of particle structures is more frequently observed in malignant melanoma cells than normal pigment cells.²⁴⁻²⁶ This observation reflects that the hierarchically aggregated structure of eumelanin may be altered via oxidative stress produced by phagosomal enzymatic activity in the process of melanomagenesis, and its biological functionality may be changed to contribute to the development and progression of malignant melanoma.

### CONCLUSIONS

Photophysical and photobiological aspects of eumelanin with relation to its biologically relevant structural alteration were explored through a well-characterized model system, pH-controlled disassembly and oxidation of natural Sepia eumelanin, in order to understand the Janus biological behavior of eumelanin. The Sepia eumelanin model that is structurally controlled by the pH-controlled disassembly and oxidation process revealed that two key optical properties of eumelanin, which provide its photo-protective function, monotonically increasing UV-vis absorption toward UV region and extremely strong non-radiative relaxation process, are governed by two characteristic non-covalent interactions: the stacking of fundamental oligomeric units and aggregation of stacked layers of oligomers. In particular, the oxidation of eumelanin subunits, which not only causes disassembly of its structure into subunits but also causes the de-stacking of stacked subunits, is a critical factor that shifts the photobiological functions of eumelanin from a photo-protective to a photoreactive species that generates ROS. These results provide clear evidence supporting the hypothesis that the disintegration of the hierarchically assembled eumelanin structure into oligomeric species, possibly induced by phagosomal enzymatic activity in the process of melanomagenesis, may trigger a switch in its biological role, such as ROS production stimulating the progression of malignant melanoma.

We believe that the present study will provide important clues to understanding full spectrum of biological aspects of eumelanin. In addition, the practical approach of manipulating the physical properties and biological aspects of eumelanin through pH-controlled disassembly and oxidation processes provides a novel platform for investigating the effects of aging-related structural disruption in various regions of melanin on its other beneficial physico-chemical properties, such as antioxidant efficiency and metal-binding capability, and determining its relevance to disease-related events, such as ocular diseases and Parkinson's disease.

Ju et al., Biomacromolecules 17:2860-2872 (2016) is herein incorporated by reference in its entirety.

### REFERENCES

(1) Sarna, T., Properties and Function of the Ocular Melanin—a Photobiophysical View. J. Photochem Photobiol. B 1992, 12, (3), 215-258.
(2) Seagle, B. L. L.; Rezai, K. A.; Kobori, Y.; Gasyna, E. M.; Rezaei, K. A.; Norris, J. R., Melanin photoprotection in the human retinal pigment epithelium and its correlation with light-induced cell apoptosis. Proc. Natl. Acad. Sci. USA. 2005, 102, (25), 8978-8983.
(3) Meredith, P.; Sarna, T., The physical and chemical properties of eumelanin. Pigm. Cell Res. 2006, 19, (6), 572-594.
(4) Felix, C. C.; Hyde, J. S.; Sarna, T.; Sealy, R. C., Melanin Photoreactions in Aerated Media—Electron-Spin Resonance Evidence for Production of Superoxide and Hydrogen-Peroxide. Biochem. Biophys. Res. Commun. 1978, 84, (2), 335-341.
(5) Korytowski, W.; Pilas, B.; Sarna, T.; Kalyanaraman, B., Photoinduced Generation of Hydrogen-Peroxide and Hydroxyl Radicals in Melanins. Photochem. Photobiol. 1987, 45, (2), 185-190.
(6) Felix, C. C.; Hyde, J. S.; Sealy, R. C., Photoreactions of Melanin—New Transient Species and Evidence for Triplet-State Involvement. Biochem. Biophys. Res. Commun. 1979, 88, (2), 456-461.
(7) Lazova, R.; Pawelek, J. M., Why do melanomas get so dark? Exp. Dermatol. 2009, 18, (11), 934-938.
(8) Moan, J.; Dahlback, A.; Setlow, R. B., Epidemiological support for an hypothesis for melanoma induction indicating a role for UVA radiation. Photochem. Photobiol. 1999, 70, (2), 243-247.
(9) Liu-Smith, F.; Dellinger, R.; Meyskens, F. L., Updates of reactive oxygen species in melanoma etiology and progression. Archives of biochemistry and biophysics 2014, 563, 51-55.
(10) Ito, S., Reexamination of the structure of eumelanin. Biochim. Biophys. Acta. 1986, 883, (1), 155-61.
(11) Cheng, J.; Moss, S. C.; Eisner, M., X-Ray Characterization of Melanins 0.2. Pigm. Cell Res. 1994, 7, (4), 263-273.
(12) Cheng, J.; Moss, S. C.; Eisner, M.; Zschack, P., X-Ray Characterization of Melanins 0.1. Pigm. Cell Res. 1994, 7, (4), 255-262.
(13) Zajac, G. W.; Gallas, J. M.; Alvaradoswaisgood, A. E., Tunneling Microscopy Verification of an X-Ray Scattering-Derived Molecular-Model of Tyrosine-Based Melanin. J. Vac. Sci. Technol. B 1994, 12, (3), 1512-1516.
(14) Zajac, G. W.; Gallas, J. M.; Cheng, J.; Eisner, M.; Moss, S. C.; Alvarado-Swaisgood, A. E., The fundamental unit of synthetic melanin: a verification by tunneling microscopy of X-ray scattering results. Biochim. Biophys. Acta. 1994, 1199, (3), 271-8.
(15) Clancy, C. M. R.; Simon, J. D., Ultrastructural organization of eumelanin from Sepia officinalis measured by atomic force microscopy. Biochemistry-Us 2001, 40, (44), 13353-13360.
(16) Nofsinger, J. B.; Simon, J. D., Radiative relaxation of Sepia eumelanin is affected by aggregation. Photochem. Photobiol. 2001, 74, (1), 31-37.
(17) Simon, J. D.; Nofsinger, J. B., Aggregation of eumelanin mitigates photogeneration of reactive oxygen species. Free Radical Bio. Med. 2001, 31, S25-S25.
(18) Nofsinger, J. B.; Forest, S. E.; Simon, J. D., Explanation for the disparity among absorption and action spectra of eumelanin. J. Phys. Chem. B 1999, 103, (51), 11428-11432.
(19) Gallas, J. M.; Zajac, G. W.; Sarna, T.; Stoner, P. L., Structural differences in unbleached and mildly-bleached synthetic tyrosine-derived melanins identified by scanning probe microscopies. Pigm. Cell Res. 2000, 13, (2), 99-108.
(20) Littrell, K. C.; Gallas, J. M.; Zajac, G. W.; Thiyagarajan, P., Structural studies of bleached melanin by synchrotron small-angle X-ray scattering. Photochem. Photobiol. 2003, 77, (2), 115-120.
(21) Fruehauf, J. P.; Trapp, V., Reactive oxygen species: an Achilles' heel of melanoma? Expert Rev. Anticancer Ther. 2008, 8, (11), 1751-1757.
(22) Simon, J. D.; Hong, L.; Peles, D. N., Insights into Melanosomes and Melanin from Some Interesting Spatial and Temporal Properties. J. Phys. Chem. B 2008, 112, (42), 13201-13217.
(23) Jimbow, K.; Quevedo, W. C.; Fitzpatrick, T. B.; Szabo, G., Some Aspects of Melanin Biology—1950-1975. J. Invest. Dermatol. 1976, 67, (1), 72-89.
(24) Rhodes, A. R.; Seki, Y.; Fitzpatrick, T. B.; Stern, R. S., Melanosomal Alterations in Dysplastic Melanocytic Nevi—a Quantitative, Ultrastructural Investigation. Cancer 1988, 61, (2), 358-369.
(25) Borovansky, J.; Mirejovsky, P.; Riley, P. A., Possible Relationship between Abnormal Melanosome Structure and Cytotoxic Phenomena in Malignant-Melanoma. Neoplasma 1991, 38, (4), 393-400.
(26) Curran, R. C.; McCann, B. G., The ultrastructure of benign pigmented naevi and melanocarcinomas in man. J. Pathol. 1976, 119, (3), 135-46.
(27) Borovansky, J.; Elleder, M., Melanosome degradation: Fact or fiction. Pigm. Cell Res. 2003, 16, (3), 280-286.
(28) Bielski, B. H. J.; Shiue, G. G.; Bajuk, S., Reduction of Nitro Blue Tetrazolium by Co2- and O-2-Radicals. J. Phys. Chem. 1980, 84, (8), 830-833.
(29) Yamakoshi, Y.; Umezawa, N.; Ryu, A.; Arakane, K.; Miyata, N.; Goda, Y.; Masumizu, T.; Nagano, T., Active oxygen species generated from photoexcited fullerene (C-60) as potential medicines: O-2(-center dot) versus O-1(2). J. Am. Chem. Soc. 2003, 125, (42), 12803-12809.
(30) Manevich, Y.; Held, K. D.; Biaglow, J. E., Coumarin-3-carboxylic acid as a detector for hydroxyl radicals generated chemically and by gamma radiation. Radiat. Res. 1997, 148, (6), 580-591.
(31) Biaglow, J. E.; Kachur, A. V., The generation of hydroxyl radicals in the reaction of molecular oxygen with polyphosphate complexes of ferrous ion. Radiat. Res. 1997, 148, (2), 181-7.
(32) Hong, L.; Simon, J. D., Current understanding of the binding sites, capacity, affinity, and biological significance of metals in melanin. J. Phys. Chem. B 2007, 111, (28), 7938-47.
(33) Nofsinger, J. B.; Forest, S. E.; Eibest, L. M.; Gold, K. A.; Simon, J. D., Probing the building blocks of eumelanins using scanning electron microscopy. Pigm. Cell Res. 2000, 13, (3), 179-184.
(34) Klegeris, A.; Korkina, L. G.; Greenfield, S. A., Autoxidation of Dopamine—a Comparison of Luminescent and Spectrophotometric Detection in Basic Solutions. Free Radical Bio. Med. 1995, 18, (2), 215-222.
(35) Pezzella, A.; Napolitano, A.; dIschia, M.; Prota, G.; Seraglia, R.; Traldi, P., Identification of partially degraded oligomers of 5,6-dihydroxyindole-2-carboxylic acid in Sepia melanin by matrix-assisted laser desorption/ionization mass spectrometry. Rapid Commun. Mass Sp. 1997, 11, (4), 368-372.
(36) Aime, S.; Fasano, M.; Terreno, E.; Groombridge, C. J., Nmr-Studies of Melanins-Characterization of a Soluble Melanin Free Acid from Sepia Ink. Pigm. Cell Res. 1991, 4, (5-6), 216-221.
(37) Meredith, P.; Powell, B. J.; Riesz, J.; Nighswander-Rempel, S. P.; Pederson, M. R.; Moore, E. G., Towards structure-property-function relationships for eumelanin. Soft Matter 2006, 2, (1), 37-44.
(38) Huijser, A.; Pezzella, A.; Sundstrom, V., Functionality of epidermal melanin pigments: current knowledge on UV-dissipative mechanisms and research perspectives. Phys. Cehm. Chem. Phys. 2011, 13, (20), 9119-9127.
(39) Tran, M. L.; Powell, B. J.; Meredith, P., Chemical and structural disorder in eumelanins: A possible explanation for broadband absorbance. Biophys. J. 2006, 90, (3), 743-752.
(40) Pezzella, A.; Panzella, L.; Crescenzi, O.; Napolitano, A.; Navaratman, S.; Edge, R.; Land, E. J.; Barone, V.; d'Ischia, M., Short-lived quinonoid species from 5,6-dihydroxyindole dimers en route to eumelanin polymers: Integrated chemical, pulse radiolytic, and quantum mechanical investigation. J Am. Chem. Soc. 2006, 128, (48), 15490-15498.
(41) Stark, K. B.; Gallas, J. M.; Zajac, G. W.; Golab, J. T.; Gidanian, S.; McIntire, T.; Farmer, P. J., Effect of stacking and redox state on optical absorption spectra of melanins-comparison of theoretical and experimental results. J. Phys. Chem. B 2005, 109, (5), 1970-1977.
(42) Pezzella, A.; Iadonisi, A.; Valerio, S.; Panzella, L.; Napolitano, A.; Adinolfi, M.; d'Ischia, M., Disentangling Eumelanin “Black Chromophore”: Visible Absorption Changes As Signatures of Oxidation State- and Aggregation-Dependent Dynamic Interactions in a Model Water-Soluble 5,6-Dihydroxyindole Polymer. J. Am. Chem. Soc. 2009, 131, (42), 15270-15275.
(43) Prampolini, G.; Cacelli, I.; Ferretti, A., Intermolecular interactions in eumelanins: a computational bottom-up approach. I. small building blocks. RSC Adv. 2015, 5, (48), 38513-38526.
(44) Chen, C. T.; Chuang, C.; Cao, J. S.; Ball, V.; Ruch, D.; Buehler, M. J., Excitonic effects from geometric order and disorder explain broadband optical absorption in eumelanin. Nat. Commun. 2014, 5.
(45) Giaimo, J. M.; Lockard, J. V.; Sinks, L. E.; Scott, A. M.; Wilson, T. M.; Wasielewski, M. R., Excited singlet states of covalently bound, cofacial dimers and trimers of perylene-3,4: 9,10-bis(dicarboximide)s. J. Phys. Chem. A 2008, 112, (11), 2322-2330.
(46) Kalinowski, J., Excimers and exciplexes in organic electroluminescence. Mater. Sci. Poland 2009, 27, (3), 735-756.
(47) Ward, W. C.; Lamb, E. C.; Gooden, D.; Chen, X.; Burinsky, D. J.; Simon, J. D., Quantification of naturally occurring pyrrole acids in melanosomes. Photochem. Photobiol. 2008, 84, (3), 700-705.
(48) Napolitano, A.; Pezzella, A.; Vincensi, M. R.; Prota, G., Oxidative-Degradation of Melanins to Pyrrole Acids—a Model Study. Tetrahedron 1995, 51, (20), 5913-5920.
(49) Sahoo, D.; Adhikary, T.; Chowdhury, P.; Chakravorti, S., Theoretical study of excited state proton transfer in pyrrole-2-carboxylic acid. Mol. Phys. 2008, 106, (11), 1441-1449.
(50) Wang, Z.; Dillon, J.; Gaillard, E. R., Antioxidant properties of melanin in retinal pigment epithelial cells. Photochem. Photobiol. 2006, 82, (2), 474-9.
(51) Geremia, E.; Corsaro, C.; Bonomo, R.; Giardinelli, R.; Pappalardo, P.; Vanella, A.; Sichel, G., Eumelanins as Free-Radicals Trap and Superoxide-Dismutase Activities in Amphibia. Comp. Biochem. Physiol. B Biochem. Mol. Biol. 1984, 79, (1), 67-69.
(52) Sarna, T.; Pilas, B.; Land, E. J.; Truscott, T. G., Interaction of Radicals from Water Radiolysis with Melanin. Biochim. Biophys. Acta. 1986, 883, (1), 162-167.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

What is claimed is:

1. A composition comprising disassembled, stacked melanin oligomers comprising 5,6-dihydroxylindole (DHI).

2. The composition of claim 1, wherein the disassembled, stacked melanin oligomers further comprise pyrrole-2,3-dicarboxylic acid (PDCA).

3. The composition of any one of claims 1-2, wherein the disassembled, stacked melanin oligomers are generated by disassembly of MelNPs.

4. The composition of any one of claims 1-3, wherein the disassembled, stacked melanin oligomers comprise 2 to about 30 oligomers layers.

5. The composition of any one of claims 1-4, wherein the disassembled, stacked melanin oligomers have a thickness of about 0.3 nm to about 16 nm.

6. The composition of any one of claims 1-5, wherein the disassembled, stacked melanin oligomers are covalently bonded to poly(ethylene glycol) (PEG).

7. The composition of claim 6, wherein the PEG has a weight average molecular weight of about 0.3 KDa to about 40 KDa.

8. The composition of any one of claims 1-7, wherein the disassembled, stacked melanin oligomers are complexed with a paramagnetic metal ion.

9. The composition of claim 8, wherein the paramagnetic metal ion is gadolinium (Gd), iron (Fe), manganese (Mn), nickel (Ni), copper (Cu), erbium (Er), europium (Eu), holmium (Ho), and/or chromium (Cr).

10. The composition of any one of claims 1-9, further comprising a magnetic resonance imaging (MRI) contrast agent.

11. A pharmaceutical composition comprising the composition of any one of claims 1-10.

12. A method of preparing a composition comprising disassembled, stacked melanin oligomers, comprising:
adding a base to melanin-like nanoparticles (MelNPs) comprising 5,6-dihydroxylindole (DHI) and disassembling the MelNPs into disassembled, stacked melanin oligomers; and
adding an acid to neutralize the disassembled, stacked melanin oligomers.

13. The method of claim 12, wherein the disassembling occurs at pH 9 or greater.

14. A method of preparing a composition comprising disassembled, stacked melanin oligomers, comprising:
adding a base to melanin-like nanoparticles (MelNPs) comprising 5,6-dihydroxylindole (DHI) to obtain a pH of greater than 10.5 and disassembling the MelNPs into disassembled, stacked melanin oligomers.

15. The method of any one of claims 12-14, wherein the MelNPs further comprise pyrrole-2,3-dicarboxylic acid (PDCA).

16. The method of any one of claims 12-15, wherein the MelNPs are synthesized from a melanin precursor of dopamine.

17. The method of any one of claims 12-16, performed under a deoxygenated and/or nitrogen purged condition.

18. The method of any one of claims 12-16, performed under an oxygenated condition.

19. The method of any one of claims 12-17, prepared in presence of poly(ethylene glycol) (PEG).

20. The method of claim 18, wherein the PEG has a weight average molecular weight of about 0.3 KDa to about 40 KDa.

21. The method of any one of claims 12-20, wherein the disassembled, stacked melanin oligomers comprise 2 to about 30 layers.

22. The method of any one of claims 12-21, wherein the disassembled, stacked melanin oligomers have a thickness of about 0.3 nm to about 16 nm.

23. The method of any one of claims 12-22, further adding a paramagnetic metal ion to the disassembled, stacked melanin oligomers.

24. The method of claim 23, wherein the paramagnetic metal ion is gadolinium (Gd), iron (Fe), manganese (Mn), nickel (Ni), copper (Cu), erbium (Er), europium (Eu), holmium (Ho), and/or chromium (Cr).

25. A method of imaging a subject, comprising:
exposing a subject to an imaging device, wherein the composition of any one of claims 1-11 has been introduced to the subject; and
detecting the disassembled, stacked melanin oligomers that are selectively accumulated in an area.

26. A method of imaging a disease in a subject, comprising:
exposing a subject to an imaging device, wherein the composition of any one of claims 1-11 has been introduced to the subject; and
detecting the disassembled, stacked melanin oligomers that are selectively accumulated in a disease area.

27. The method of claim 26, wherein the disease is a tumor.

28. The method of any one of claims 25-27, wherein the imaging device is MRI.
